(19) United States
(12) Patent Application Publication
(10) Pub. No.: US 2019/0316133 A1
(43) Pub. Date: Oct. 17, 2019

(54) Anti-Fibrinogen Aptamers and Uses Thereof

(71) Applicant: Laboratoire Francais du Fractionnement et des Biotechnologies, Les Ulis (FR)

(72) Inventor: Alexander SEIFERT, Paris (FR)

(73) Assignee: Laboratoire Francais du Fractionnement et des Biotechnologies, Les Ulis (FR)

(21) Appl. No.: 16/312,457

(22) PCT Filed: Jul. 6, 2017
(86) PCT No.: PCT/EP2017/066952

§ 371 Date: Dec. 21, 2018

(30) Foreign Application Priority Data
Jul. 6, 2016 (FR) 1656479
Jul. 28, 2016 (EP) 16305984.0
Jul. 28, 2016 (EP) 16305985.3

(51) Int. Cl. C12N 15/115; C12Q 1/68; G01N 33/50; C07H 21/04

(52) U.S. Cl. CPC C12N 15/115; C12Q 1/68; C12N 2320/50; C07H 21/04; C12N 2310/16; G01N 33/5002

(57) ABSTRACT

The invention relates to aptamers which specifically bind to fibrinogen and their use in the purification of said protein.

### FIELD OF THE INVENTION

The invention relates to affinity ligands which specifically bind to fibrinogen and their use in the purification of said protein.

### BACKGROUND OF THE INVENTION

Fibrinogen is a plasma large soluble and complex glycoprotein. Fibrinogen exists as a dimer of three polypeptide chains the Aα (66.5 kD), Bβ. (52 kD) and γ (46.5 kD) which are linked through 29 disulphide bonds and result in a molecule with a molecular weight of 340 kD. Fibrinogen has a trinodal structure: a central nodule, called the E domain, containing the N-termini of all 6 chains which include the fibrinopeptides and two distal nodules, called D domains, containing the C-termini of the Aα, Bβ and γ chains. Fibrinogen is synthesized in the liver by the hepatocytes and its concentration in blood plasma is about 200-400 mg/dL. Fibrinogen plays a key role in blood clotting cascade. Fibrinogen is proteolytically cleaved at the amino terminus of the Aα and Bβ releasing fibrinopeptides A and B, and converted to fibrin monomers, the building block of hemostatic plug, by thrombin (factor IIa). The resulting fibrin monomers self-assemble into fibrin polymers which are crosslinked by activated Factor XIII. Fibrinogen is also involved in other biological process, such as inflammation and wound healing.

Deficiencies in fibrinogen can lead to clotting disorders characterized by an increased tendency of bleedings. The availability of fibrinogen in purified form is of high therapeutic interest. Indeed, injectable forms of purified fibrinogen are used in the treatments of congenital or acquired deficiencies in fibrinogen (hypo-, dys- or afibrinogenaemia). Fibrinogen is also used in the management of post-traumatic or post-surgical acute hemorrhages or in the management of fibrinogen deficiency resulting from acute renal failure.

The main source of fibrinogen is human plasma. Various methods for the purification of fibrinogen have been described in the state of the art. Most of them are based on precipitation techniques such as cryoprecipitation (Sparrow, 2011, Methods Mol Biol.; 728:259-65) or conventional precipitation (WO 2008121330) and lead to relatively pure products but which nevertheless comprise other plasma proteins as contaminants such as plasminogen, tPA, factor XIII and fibronectin. Indeed, fibrinogen has a propensity for binding other plasma proteins, which are thus often co-purified during precipitation techniques. The purification of fibrinogen from human plasma by column chromatography has been also described in Kuyas et al., (Thrombosis and Haemostasis, 1990, 63, 439-444) and in Takebe (Thrombosis and Haemostasis, 1995, 73, 662-667), which illustrate the use of affinity supports comprising either peptide ligands or anti-fibrinogen antibodies. However, the use of such stationary supports at the industrial scale is prohibited because of their cost and the lability of the grafted ligands. The production of fibrinogen in milk of transgenic animals and its subsequent purification has been also described, for instance in PCT applications WO9523868 and WO200017239. However, the purification of fibrinogen from milk is still a real challenge because the final product must be devoid of any non-human contaminating proteins which may be antigenic. There is thus a need for alternative methods for the purification for fibrinogen.

### SUMMARY OF THE INVENTION

The invention relates to an aptamer which specifically binds to fibrinogen in a pH-dependent manner, preferably which does not bind to fibrinogen at a pH higher than 7.0 but specifically binds to fibrinogen at an acid pH, for instance at a pH value selected from 6.0 to 6.6.

The invention also relates to an aptamer capable of specifically binding to fibrinogen, wherein
a. Said aptamer comprises a polynucleotide having at least 70% of sequence identity with the nucleotide sequence of SEQ ID NO: 66, or
b. Said aptamer comprises the nucleotide moiety of formula (III):

5′-[SEQ ID NO: 79]-[X1]-[SEQ ID NO: 77]-[X2]- [SEQ
ID NO: 78]-3′ (III)

wherein:
[X2] and [X1] independently denote a nucleotide or an oligonucleotide of 2 to 5 nucleotides in length, preferably of 2 or 3 nucleotides in length,
[SEQ ID NO:77] is an oligonucleotide of SEQ ID NO:77 (namely GTTGGTAGGG),
[SEQ ID NO:78] is an oligonucleotide of SEQ ID NO:78 (namely GGTGTAT) and [SEQ ID NO:79] is an oligonucleotide of SEQ ID NO:79 (namely TGT).

In some embodiments, the aptamer of the invention has at least 70% of sequence identity with a nucleotide sequence selected from the group consisting of SEQ ID NO:66, SEQ ID NO:67, SEQ ID NO:68, SEQ ID NO:69, SEQ ID NO:70, SEQ ID NO:71, SEQ ID NO:72, SEQ ID NO:73 and SEQ ID NO:74.

In other embodiments, the aptamer of the invention can be of formula (I)

5′-[NUC1]ₘ[CENTRAL]-[NUC2]ₙ-3′

### Wherein

n and m are integers independently selected from 0 and 1,

[NUC1] is a polynucleotide comprising from 2 to 40 nucleotides,
[NUC2] is a polynucleotide comprising from 2 to 40 nucleotides, and
[CENTRAL] is a polynucleotide having at least 70% of sequence identity with a nucleotide sequence selected from the group consisting of SEQ ID NO:68, SEQ ID NO:69, SEQ ID NO:70, SEQ ID NO:71, SEQ ID NO:72, SEQ ID NO:73, SEQ ID NO:74, SEQ ID NO:94 and SEQ ID NO:95.

In some embodiments, the aptamer of the invention comprises a polynucleotide of SEQ ID NO:66, or differs from SEQ ID NO:66 in virtue of from 1 to 14 nucleotide modifications at nucleotide positions selected from 1, 2, 11-25, 32-35, 42, 45-47, 50 and 54-58, the numbering referring to nucleotide numbering in SEQ ID NO:66.

In another embodiment, the aptamer of the invention comprises a polynucleotide selected from the group consisting of SEQ ID NO:66, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:88, SEQ ID NO:89, SEQ ID NO:90, SEQ ID NO:91, SEQ ID NO:92 and SEQ ID NO:93.

In a particular aspect, the aptamer of the invention comprises at least 70% of sequence identity with a nucleotide sequence selected from the group consisting of SEQ ID NO:1-NO:67. For instance, the aptamer of the invention can specifically bind to human plasma fibrinogen or recombinant human fibrinogen.

Another object of the invention is an affinity ligand capable of specifically binding to fibrinogen which comprises an aptamer as defined above and at least one moiety selected from a mean of detection and a mean of immobilization onto a support.

The invention also relates to a solid affinity support comprising thereon a plurality of affinity ligands or a plurality of aptamers as defined above.

Another object of the invention is a method for preparing a purified fibrinogen composition from a starting fibrinogen-containing composition comprising:
a. contacting said starting composition with an affinity support as defined above, in conditions suitable to form a complex between (i) the aptamers or the affinity ligands immobilized on said support and (ii) fibrinogen
b. releasing fibrinogen from said complex, and
c. recovering a purified fibrinogen composition.

In some embodiments, step a) is performed at a pH lower than 7.0, preferably at a pH from 6.0 to 6.6, and step b) is performed at a pH above 7.0, preferably at pH from 7.2 to 7.6.

In some additional or alternate embodiments, steps a)-c) are performed by chromatography technology.

The invention also relates to the use of an aptamer, an affinity ligand or an affinity support as defined above in the purification of fibrinogen, in the detection of fibrinogen, in a blood plasma fractionation process or in the preparation of a composition comprising fibrinogen which is stable in liquid form.

In a further aspect, the invention relates to a blood plasma fractionation process comprising:

(a) an affinity chromatography step to recover fibrinogen wherein the affinity ligand is an aptamer which specifically binds to fibrinogen,
(b) an affinity chromatography step to recover immunoglobulins (Ig) wherein the affinity ligand is an aptamer which specifically binds to immunoglobulins, and
(c) optionally a purification step of albumin, wherein steps (a), (b) and (c) can be performed in any order.

The invention also relates to a liquid composition comprising fibrinogen which is stable, said composition being obtainable by a method comprising the steps of:
providing a blood plasma or a cryosupernatant fraction of blood plasma,
purifying said blood plasma or said cryosupernatant fraction of blood plasma by separation on affinity chromatography gel using an affinity ligand preferably selected from an anti-fibrinogen aptamer as defined in herein,
collecting the purified adsorbed fraction comprising fibrinogen, and
optionally, adding pharmaceutically acceptable excipients, preferably arginine and/or citrate such as citrate salt.

### BRIEF DESCRIPTION OF THE DRAWINGS

FIG. 1A shows the predicted secondary structure of aptamer of SEQ ID NO: 1. The nucleotides belonging to the core sequence (SEQ ID No 66) are highlighted in grey

FIG. 2A shows the predicted secondary structure of aptamer of SEQ ID NO:58. The nucleotides belonging to the core sequence (SEQ ID No 67) are highlighted in grey. The framed loop corresponds to the region of the aptamer comprising the consensus moiety of formula (III).

FIG. 2B shows the alignments of the core sequences of SEQ ID NO:67-74. The framed parts of the sequences comprise the consensus moiety of formula (III).

FIGS. 3-4 show the binding properties of some aptamers directed against human fibrinogen obtained by the method of the invention

FIG. 3A shows the SPR binding curves of human plasma fibrinogen present at a concentration from 125 nM to 1000 nM on SEQ ID NO:66 (the core sequence of SEQ ID NO:1) immobilized on a chip. Each solution of human plasma fibrinogen was injected at pH 6.3 whereby a complex was formed in a dose-dependent manner as evidenced by the increase of the signals depending on the concentration of fibrinogen. The injection of a buffer solution at pH 6.3 comprising 0.5 M NaCl did not significantly induce the elution of human plasma fibrinogen. Fibrinogen was then released from the complex by an elution buffer at pH 7.40. The solid support was then regenerated by injecting a solution of NaOH at 50 mM. X-axis: time in s. Y-axis: SPR response in arbitrary scale.

FIG. 3B shows the SPR binding curves of transgenic fibrinogen present at a concentration from 125 nM to 1000 nM on SEQ ID NO:66 (the core sequence of SEQ ID NO:1) immobilized on a chip. Each solution of transgenic fibrinogen was injected at pH 6.3 whereby a complex was formed in a dose-dependent manner as evidenced by the increase of the signals depending on the concentration of fibrinogen. The injection of a buffer solution at pH 6.3 comprising 0.5 M NaCl did not significantly induce the elution of transgenic fibrinogen. Fibrinogen was then released from the complex by an elution buffer at pH 7.40. The solid support was then regenerated by injecting a solution of NaOH at 50 mM. X-axis: time in s. Y-axis: SPR response in arbitrary scale

FIG. 3C shows the SPR binding curves of human plasma fibrinogen present at a concentration from 125 nM to 1000 nM on SEQ ID NO:67 (the core sequence of SEQ ID NO:58) immobilized on a chip. Each solution of human plasma fibrinogen was injected at pH 6.3 whereby a complex was formed in a dose-dependent manner as evidenced by the increase of the signals depending on the concentration of fibrinogen. The injection of a buffer solution at pH 6.3 comprising 1 M NaCl did not considerably induce the elution of human plasma fibrinogen. Fibrinogen was then released from the complex by an elution buffer at pH 7.40 and containing MgCl₂ at 2M. The solid support was then regenerated by injecting a solution of NaOH at 50 mM. X-axis: time in s. Y-axis: SPR response in arbitrary scale.

FIG. 3D shows the SPR binding curves of transgenic fibrinogen present at a concentration from 125 nM to 1000 nM on SEQ ID NO:67 (the core sequence of SEQ ID NO:58) immobilized on a chip. Each solution of transgenic fibrinogen was injected at pH 6.3 whereby a complex was formed in a dose-dependent manner as evidenced by the increase of the signals depending on the concentration of fibrinogen. The injection of a buffer solution at pH 6.3 comprising 1 M NaCl did not considerably induce the elution of transgenic fibrinogen. Fibrinogen was then released from the complex by an elution buffer at pH 7.40 and containing MgCl₂ at 2M. The solid support was then regenerated by injecting a solution of NaOH at 50 mM. X-axis: time in s. Y-axis: SPR response in arbitrary scale.

FIG. 4A shows SPR sensograms illustrating the pH dependency of binding of fibrinogen to immobilised aptamer SEQ ID NO:66 (the core sequence of SEQ ID NO:1). Plasmatic Fibrinogen is injected at different pH, after sample injection a running buffer at pH 6.30 is passed over the flow cell in every run. The highest binding level is obtained for pH 6.30. The binding level decreases when pH increases. X-axis: time in s. Y-axis: SPR response in arbitrary scale.

FIG. 4B shows SPR sensograms illustrating the pH dependency of binding affinity of aptamer of SEQ ID NO: 67 (the core sequence of SEQ ID NO:58) to human plasma fibrinogen. No binding is observed for pH higher than 6.8. X-axis: time in s. Y-axis: SPR response in arbitrary scale.

FIG. 4C shows the binding curve of human plasma fibrinogen (sensorgram) for aptamers of SEQ ID NO:60 and SEQ ID NO:65 (belonging to the second subgroup of aptamers of the invention) immobilized on a chip, obtained by SPR technology. Purified human plasma fibrinogen (250 nM) was injected at pH 6.3, whereby a complex was formed as evidenced by the increase of the signal. The injection of a buffer solution at pH 6.3 comprising 0.5 M NaCl did not significantly induce the elution of human plasma fibrinogen. The solid support was then regenerated by injecting a solution of NaOH at 50 mM. X-axis: time in s. Y-axis: SPR response in arbitrary scale.

FIG. 5A shows the chromatographic profile for the purification of fibrinogen from plasma on an affinity support grafted with aptamer of SEQ ID NO:66. Y-axis: absorbance at 280 nm. X-axis: in mL

FIG. 5B shows the picture of the electrophoresis gels after coomassie blue staining in non-reduced conditions. From left to right: 1: plasma, 2: fraction from the plasma which was not retained on the stationary phase, 3: elution fraction containing fibrinogen obtained from the chromatography of plasma, 4: fraction obtained after regeneration of the stationary support, and 5: molecular weight markers. The purity of the elution fraction for fibrinogen was more than 95% as compared to the total amount of proteins contained in the fraction. The affinity support used in chromatography was grafted with aptamers of SEQ ID NO:66.

FIG. 6A shows the chromatographic profile for the purification of fibrinogen from plasma on an affinity support grafted with aptamer of SEQ ID NO:67. Y-axis: absorbance at 280 nm. X-axis: in mL

FIG. 6B shows the picture of the electrophoresis gels after coomassie blue staining in non-reduced conditions. From left to right: 1: plasma, 2: fraction from the plasma which was not retained on the stationary phase, 3: fraction obtained after washing of the stationary support, 4: elution fraction containing fibrinogen obtained from the chromatography of plasma, and 5: molecular weight markers. The purity of the elution fraction for fibrinogen was of least 95% as compared to the total amount of proteins contained in the fraction. The affinity support used in chromatography was grafted with aptamers of SEQ ID NO:67.

FIG. 7A shows the chromatographic profile obtained for the purification of semi-purified fibrinogen on an affinity support grafted with aptamer of SEQ ID NO:66. Y-axis: absorbance at 280 nm. X-axis: in mL

FIG. 7B shows the chromatographic profile obtained for the purification of semi-purified fibrinogen on an affinity support grafted with aptamer of SEQ ID NO:67. Y-axis: absorbance at 280 nm. X-axis: in mL.

FIG. 7C shows the analysis of the fractions by SDS-PAGE in reduced and non-reduced conditions, with AgNO₃ staining, of the elution fractions obtained by purification of intermediate fibrinogen on the affinity supports. Lane 1: molecular weight standard. Lane 2: Fibrinogen intermediate (starting material), Lane 3: Elution fraction obtained with affinity support no 1 (aptamers of SEQ ID NO:66), Lane 4: Elution fraction obtained with affinity support no 1 (aptamers of SEQ ID NO:67) FIG. 7D shows the analysis of the fractions by SDS-PAGE in reduced and non-reduced conditions, with coomassie staining, of the elution fractions obtained by purification of intermediate fibrinogen on the affinity supports. Lane 1: molecular weight standard. Lanes 2 and 3: Fibrinogen intermediate (starting material), Lanes 4 and 5: Elution fraction obtained with affinity support no 1 (aptamers of SEQ ID NO:66), Lanes 6 and 7: Elution fraction obtained with affinity support no 1 (aptamers of SEQ ID NO:67). NR: non reduced. R: Reduced.

FIG. 8 shows the SELEX protocol used to identify aptamers directed against human fibrinogen.

FIG. 9 shows the competitive binding of immobilized aptamer of SEQ ID NO:66 to injected fibrinogen in presence of aptamer variants. Here, the higher the affinity of the variant for fibrinogen (as compared to aptamer of SEQ ID NO:66) the lower the response during the injection of the variant/fibrinogen mixture. Variants of SEQ ID NO:66 comprising one of the following deletion combinations (i) 1/2, (ii) 19/20/21, (iii) 18/19/20/21, (iv) 15/16/19/20 and (v) 14/15/16/20/21/22 showed a high affinity for fibrinogen.

FIG. 10A shows the binding curves of human plasmatic and transgenic fibrinogen (sensorgram) for an aptamer from Base Pair Biotechnologies (reference 6F01 oligo#370) immobilized on a sensor chip, obtained by SPR technology. Human plasmatic and transgenic fibrinogen (1000 nM) was injected at pH 7.40 using the Base Pair Biotechnologies recommended buffer. Very low binding levels were observed for human plasmatic and transgenic fibrinogen. The solid support was then regenerated by injecting a solution of NaOH at 50 mM. X-axis: time in s. Y-axis: SPR response in arbitrary scale.

FIG. 10B shows the binding curves of human plasmatic fibrinogen (sensorgram) for aptamer SEQ ID NO:67 (A.5-2.9) of the invention and for the aptamer from Base Pair Biotechnologies (reference 6F01 oligo#370) immobilized on a sensor chip, obtained by SPR technology. Human plasmatic fibrinogen (1 μM) was injected at pH 6.3. The binding of plasmatic human fibrinogen on the aptamer of the invention was significantly higher than that on the aptamer from Base Pair Biotechnologies. The injection of a buffer solution at pH 6.3 comprising 1 M NaCl did not significantly induce the elution of human plasma fibrinogen in the case of the aptamer of the Invention. By contrast, fibrinogen is eluted in the case of the aptamer 6F01 oligo#370 which suggests that the interaction between said aptamer and plasmatic human fibrinogen are weak and non-specific.

FIG. 10C shows the binding curves of human plasmatic fibrinogen (sensorgram) for 3 aptamers (aptamers 85A, 121A and 121B) described in the supplementary data of Li et al. (J Am Chem Soc, 2008, 130 (38):12636-12638) immobilized on a sensor chip, obtained by SPR technology. Human plasmatic fibrinogen (1000 nM) was injected at pH 7.40, as recommended by Li. et al. Very low binding levels were observed for human plasmatic and transgenic fibrinogen. The solid support was then regenerated by injecting a solution of NaOH at 50 mM. X-axis: time in s. Y-axis: SPR response in arbitrary scale.

### REMARKS

MBS buffer refers to 50 mM MOPS/150 mM NaCl

MBS 1M NaCl buffer refers to 50 mM MOPS/1M NaCl

MBS-M5 buffer refers to: 50 mM MOPS pH 6.30/150 mM NaCl/5 mM MgCl₂

MBS-M5 0.5M NaCl buffer refers to 50 mM MOPS pH 6.30/0.5M NaCl/5 mM MgCl₂

### DETAILED DESCRIPTION OF THE INVENTION

Aptamers which potentially bind to fibrinogen have been described in the prior art. PCT application, WO2010/019847 describes aptamers directed against fibrinogen and fibrin and comprising at least one nucleotide having a boronic moiety (i.e. a boronic acid-modified nucleotide). Said aptamers bind to a glycosylation site of fibrinogen and may be useful as anticoagulants. US patent application 2013-0245243 in the name of Base Pair Technologies describe several potential anti-fibrinogen aptamers, but does not provide any evidence showing the actual affinity and specificity of these aptamers for fibrinogen.

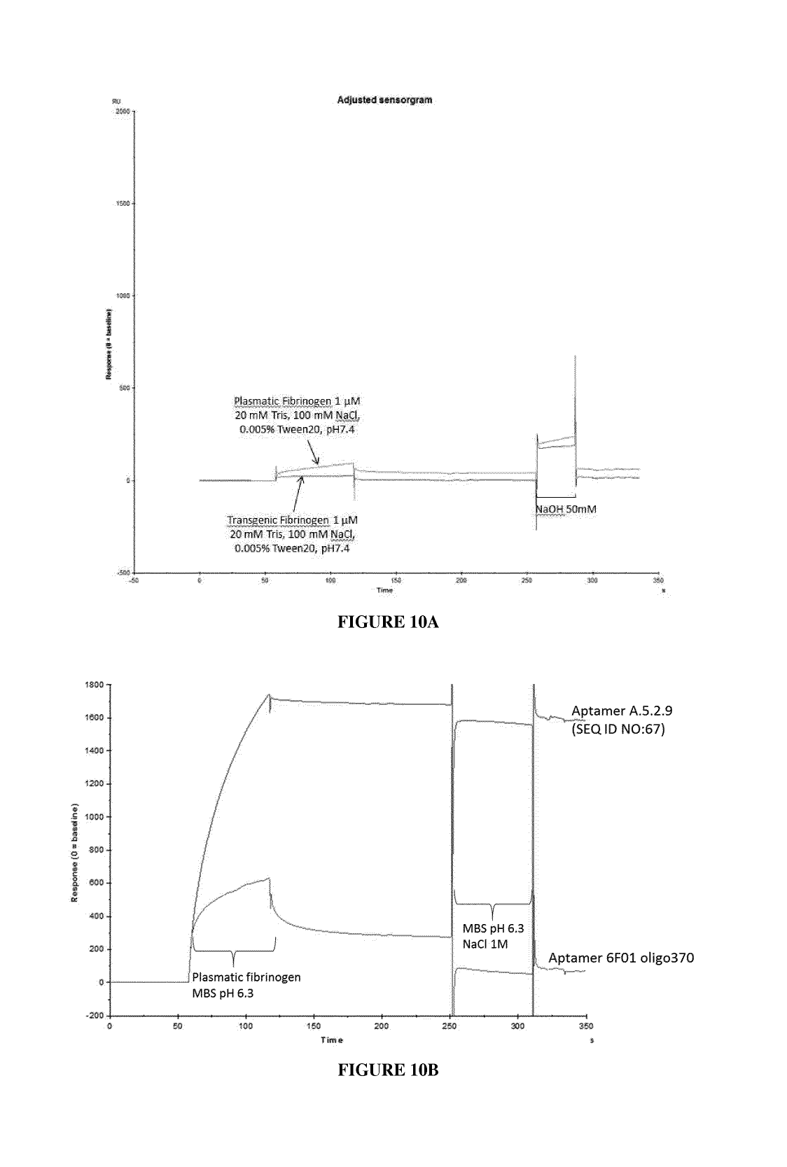

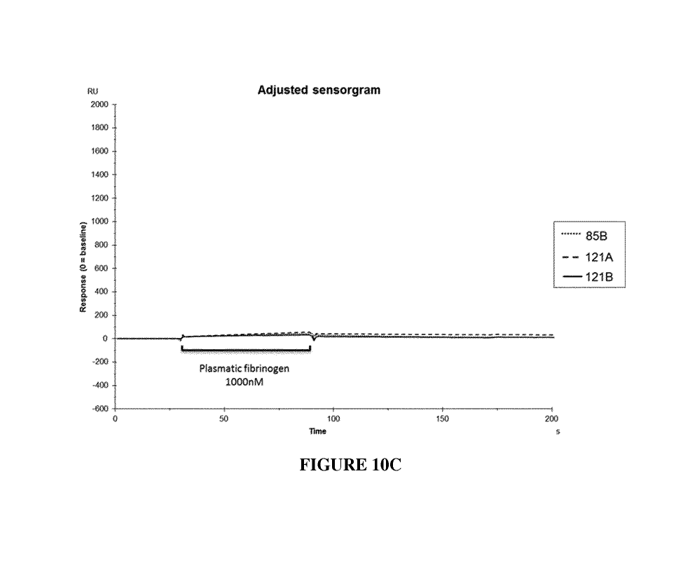

Base Pair Biotechnologies also markets an aptamer presented as anti-fibrinogen ligand (reference 6F01 oligo#370) for research use only. The Applicants investigated the ability of said aptamer to be used as affinity ligands for the purification of fibrinogen and IgGs. The experiments performed by the Applicant demonstrated that said aptamers did not have binding properties suitable for use as affinity ligands for purification. As shown in FIG. 10, the anti-fibrinogen aptamer marketed by Base Pair Biotechnologies (reference 6F01 oligo#370) displayed very low binding to both transgenic and human fibrinogen, when the binding buffer recommended by the manufacture was used (FIG. 10A). At pH 6.3, the binding of this aptamer with plasmatic fibrinogen was also low and corresponded to non-specific interactions (FIG. 10B). This low binding capacity precludes the use of the aptamer 6F01 oligo#370 as affinity ligand in purification process. Similar results were obtained for aptamers described in Li et al. (Supra) (see FIG. 10C).

The Applicant performed his own research and identified a new family of aptamers directed against fibrinogen. This new family of aptamers was identified by an in-house SELEX process conceived by the Applicant. These aptamers were shown to specifically bind both transgenic and plasma human fibrinogen, regardless the glycosylation status of the protein. The aptamers identified by the Applicant display unique properties in terms of binding. In particular, the aptamers of the invention bind to fibrinogen in a pH-dependent manner. Noteworthy they display increased binding affinity for fibrinogen at a slightly acid pH such as a pH of about 6.3 as compared to a pH higher than 7.0 such as 7.4. Such properties are particularly suitable for use in affinity chromatography because the formation of the complex between the protein to purify, namely fibrinogen, and the aptamer, and the subsequent release of the protein from the complex can be controlled by modifying the pH of the elution buffer. In particular, the release of fibrinogen from the complex can be performed in mild conditions of elution, which are not likely to alter the properties of the protein.

The aptamers of the invention can be also used as ligands for diagnostic and detection purposes, even in complex medium such as plasma.

Aptamers of the Invention

Accordingly, the invention relates to an aptamer directed against fibrinogen, i.e. able to specifically bind to fibrinogen. The aptamers of the invention bind to fibrinogen in a pH-dependent manner. Preferably, the aptamers of the invention do not bind to fibrinogen at a pH higher than 7.0 and bind to fibrinogen at an acid pH, for instance at a pH value selected from 6.0 to 6.6, such as pH 6.3±0.1.

Preferably, the aptamers of the invention are suitable as affinity ligands in the purification of a fibrinogen, for instance by chromatography.

As used herein, an “aptamer” (also called nucleic aptamer) refers to a synthetic single-stranded polynucleotide typically comprising from 20 to 150 nucleotides in length and able to bind with high affinity a target molecule. The aptamers are characterized by three-dimensional conformation(s) which may play a key role in their interactions with their target molecule. Accordingly, the aptamer of the invention is capable of forming a complex with fibrinogen. The interactions between an aptamer and its target molecule may include electrostatic interactions, hydrogen bonds, and aromatic stacking shape complementarity. “An aptamer specifically binds to its target molecule” means that the aptamer displays a high affinity for the target molecule. The dissociation constant (Kd) of an aptamer for its target molecule is typically from 10⁻⁶ to 10⁻¹² M. The term “specifically binding” is used herein to indicate that the aptamer has the capacity to recognize and interact specifically with its target molecule, while having relatively little detectable reactivity with other molecules which may be present in the sample. Preferably, the aptamer specifically binds to its target molecule if its affinity is significantly higher for the target molecule, as compared to other molecules, including molecules structurally close to the target molecule.

For instance, an aptamer might be able to specifically bind to a human protein while displaying a lower affinity for a homolog of said human protein.

As used herein, “an aptamer display a lower affinity for a given molecule as compared to its target molecule” or “an aptamer is specific to its target molecule as compared to a given molecule” means that the Kd of the aptamer for said given molecule is at least 5-fold, preferably, at least 10, 20, 30, 40, 50, 100, 200, 500, or 1000-fold higher than the Kd of said aptamer for the target molecule.

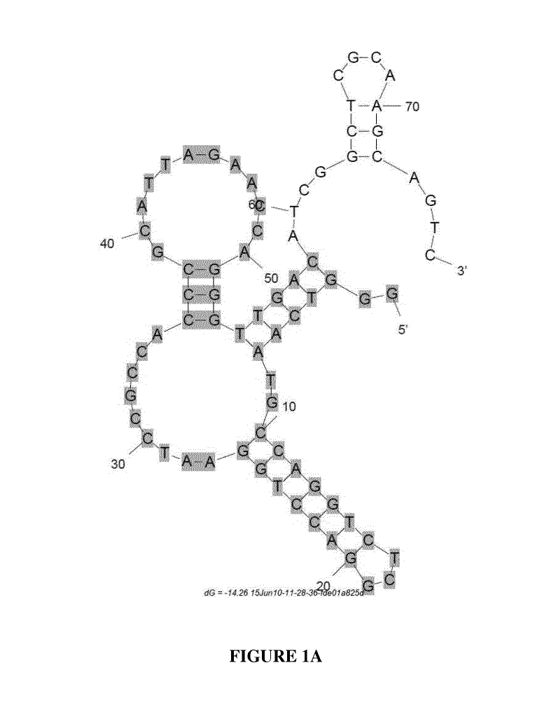

The aptamers may be a deoxyribonucleic acid (DNA) or a ribonucleic acid (RNA). The aptamers can comprise one or several chemically-modified nucleotides. Chemically-modified nucleotides encompass, without being limited to 2′-amino, or 2′ fluoro nucleotides, 2′-ribopurine, phosphoramidite, locked nucleic acid (LNA), boronic acid-modified nucleotides, 5-iodo or 5-bromo-uracil, and 5-modified deoxyuridine such as benzyl-dU, isobutyl-dU, and naphtyl-dU. For 5-modified deoxyuridine, one can refer to Rohloff et al., Molecular Therapy-Nucleic acids, 2014, 3, e201 (see FIG. 1 page 4), the disclosure of which being incorporated herein by reference. In some embodiments, the aptamer of the invention is devoid of any boronic acid-modified nucleotides, in particular those taught in WO2010/019847. In some other embodiments, the aptamer of the invention is devoid of any 5-modified deoxyuridine.

In certain embodiments, the aptamer may comprise a modified nucleotide at its 3′-extremity or/and 5′-extremity only (i.e. the first nucleotide and/or the last nucleotide of the aptamer is/are the sole chemically-modified nucleotide(s)). Preferably, said modified nucleotide may enable the grafting of the aptamer onto a solid support, or the coupling of said aptamer with any moiety of interest (e.g. useful for detection or immobilization).

Once the sequence of the aptamer is identified, the aptamer can be prepared by any routine method known by the skilled artisan, namely by chemical oligonucleotide synthesis, for instance in solid phase.

As used herein, “an aptamer directed to fibrinogen”, “an aptamer directed against fibrinogen” or “an anti fibrinogen aptamer” refers to a synthetic single-stranded polynucleotide which specifically binds to fibrinogen.

As used herein, the term “fibrinogen” refers to any protein having the amino acid sequence of a wild-type fibrinogen and variants thereof, regardless the glycosylation state. The term “fibrinogen” encompasses any isoforms or allelic variants of fibrinogen, as well as any glycosylated forms, non-glycosylated forms or post-translational modified forms of fibrinogen.

As used herein, a variant of a wild-type fibrinogen refers to a protein having at least 80% of sequence identity, preferably at least 85%, 90%, or 95% of sequence identity with said wild-type fibrinogen and which displays a similar biological activity as compared to said wild-type fibrinogen. For instance, the clotting activity of the fibrinogen can be measured by van Clauss coagulation method. The fibrinogen variant may have an increased or a decreased biological activity as compared to the corresponding wild-type fibrinogen.

In some embodiments, the fibrinogen refers to a protein having the amino acid sequence of a human wild-type fibrinogen or a variant thereof. Said fibrinogen may be a human plasma fibrinogen, a recombinant or transgenic human fibrinogen. In some embodiments, the aptamer of the invention is able to bind a human fibrinogen, regardless its glycosylation. For instance an aptamer of the invention may be able to specifically bind to human plasma fibrinogen and recombinant human fibrinogen, for instance a recombinant fibrinogen obtained from a transgenic multicellular organism or a recombinant fibrinogen obtained from a recombinant host cell.

The aptamers of the invention may be able to specifically bind to fibrinogen at a slightly acid pH, for instance at pH 6.3.

Preferably, the aptamer of the invention displays a constant dissociation (Kd) for a human plasma fibrinogen or for a transgenic human fibrinogen of at most 10⁻⁶ M. Typically, the Kd of the aptamers of the invention for human fibrinogen may be from 1.10⁻¹² M to 1.10⁻⁶ M at a pH of about 6.3. Kd is preferably determined by surface plasmon resonance (SPR) assay in which the aptamer is immobilized on the biosensor chip and fibrinogen is passed over the immobilized aptamers, at a pH of interest, and at various concentrations, under flow conditions leading to the measurements of kₒₙ and k_{off} and thus Kd. One can refer to the protocol provided in Example 1.

In some embodiments, the aptamer of the invention is specific to a human fibrinogen as compared to a non-human fibrinogen.

In some other embodiments, the aptamer of the invention is specific to human fibrinogen as compared to other proteins present in plasma, such as clotting factors. Preferably, the aptamer of the invention specifically binds to fibrinogen as compared to factor FII, FXI or XIII. In additional or alternative embodiments, the aptamer of the invention specifically binds to fibrinogen as compared to fibronectin. In additional or alternative embodiments, the aptamer of the invention specifically binds to fibrinogen as compared to plasminogen.

Preferably, the aptamer of the invention does not bind to fibrinogen at a pH of 7.0 or above. The inability of the aptamer of the invention to bind to fibrinogen at a pH of 7.0 and above can be determined typically by SPR as described in Example 1. In the protocol of Example 1, an absence of binding is shown by the fact that the SPR signal remains in the baseline after the injection of fibrinogen in a buffered tampon at the pH of interest.

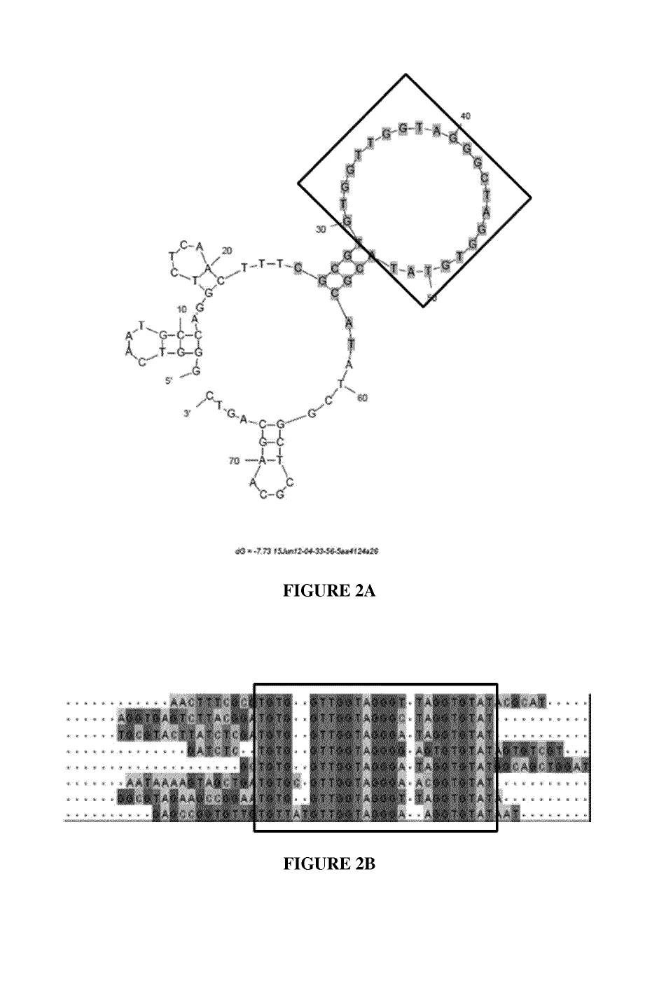

In a certain aspect of the invention, the aptamers may be characterized by the presence of a specific moiety in their conformation. For instance, the aptamers of the invention may comprise a moiety as shown in FIG. 1A or FIG. 2A. Without to be bound by any theory, the Applicant believes that the presence of said two-dimensional conformation may be involved in the specific interactions with fibrinogen.

The presence of said specific conformational moiety may result from the presence of a specific polynucleotide (called “core polynucleotide” or “core sequence”) within the aptamer sequence. As used herein, a “core sequence” of a given aptamer typically comprises, or refers to, the minimal sequence issued from said aptamer able to bind a fibrinogen.

By studying the aptamers identified by his own research, the Applicant identified several core sequences of interest, among others the polynucleotides of SEQ ID NO: 66 and SEQ ID NO:67. The Applicant further determined the consensus sequence moieties in SEQ ID NO: 58-65. As shown in FIG. 2B, aptamers of SEQ ID NO: 58-65 comprise two consensus moieties in their sequences, namely GTTGGTAGGG (SEQ ID NO:77) which is upstream of GGTGTAT (SEQ ID NO:78). These consensus moieties are located in a region of the aptamers which forms a loop as evidenced in FIG. 2A for the aptamer of SEQ ID NO:58. Without to be bound to any theory, the Applicant believes that this conformational moiety may play a role in the binding of said aptamers to fibrinogen.

In a certain aspect, the invention relates to an aptamer capable of specifically binding to fibrinogen and having one of the following features:
Said aptamer comprises a polynucleotide having at least 70%, e.g. at least 75%, 80%, 85%, 90%, or 95% of sequence identity with the nucleotide sequence of SEQ ID No 66, or Said aptamer comprises the nucleotide moieties GTTGGTAGGG (SEQ ID NO:77) and

GGTGTAT (SEQ ID NO:78), wherein the moiety of SEQ ID NO:77 is preferably upstream to SEQ ID NO:78.

In some embodiments, the aptamer of the invention is capable of specifically binding to fibrinogen and has one of the following features:
Said aptamer comprises a polynucleotide having at least 70% of sequence identity with the nucleotide sequence of SEQ ID No 66, or
Said aptamer comprises the nucleotide moiety of formula (III):

5′-[SEQ ID NO: 79]-[X1]-[SEQ ID NO: 77]-[X2]- SEQ
ID NO: 78]-3′ (III)

wherein:
[X2] and [X1] independently denote a nucleotide or an oligonucleotide of 2 to 5 nucleotides in length, preferably of 2 or 3 nucleotides in length,
[SEQ ID NO:77] is an oligonucleotide of SEQ ID NO:77 (namely GTTGGTAGGG),
[SEQ ID NO:78] is an oligonucleotide of SEQ ID NO:78 (namely GGTGTAT) and
[SEQ ID NO:79] is an oligonucleotide of SEQ ID NO:79 (namely TGT).

In a particular embodiment, the aptamer of the invention comprises a polynucleotide which:
has at least 70% of sequence identity with at least one nucleotide sequence selected from the group consisting of SEQ ID NO:67, SEQ ID NO:68, SEQ ID NO:69, SEQ ID NO:70, SEQ ID NO:71, SEQ ID NO:72, SEQ ID NO:73, SEQ ID NO:74, and SEQ ID NO:95 and
comprises the nucleotide moiety of formula (III) as defined above.

For instance, the aptamer of the invention may comprise a polynucleotide which has at least 70% of sequence identity with SEQ ID NO:67 and which comprises the nucleotide moiety of formula (III).

In another aspect, the invention relates to an aptamer capable of specifically binding to fibrinogen and comprising a polynucleotide having at least 70% of sequence identity with SEQ ID No 66 or SEQ ID NO:67.

As used herein, a sequence identity of at least 70% encompasses a sequence identity of at least 75%, 80%, 85%, 86%, 87%, 88%, 89%, 90%, 91%, 92%, 93%, 94%, 95%, 96%, 97%, or 98%.

The “percentage identity” between two nucleotide sequences (A) and (B) may be determined by comparing the two sequences aligned in an optimal manner, through a window of comparison. Said alignment of sequences can be carried out by well-known methods, for instance, using the algorithm for global alignment of Needleman-Wunsch. Once alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two nucleic acid sequences, one can use, for example, the tool “Emboss needle” for pairwise sequence alignment of providing by EMBL-EBI and available on http://www.ebi.ac.uk/Tools/psa/emboss_needle/nucleotide.html using default settings: (I) Matrix: DNAfull, (ii) Gap open: 10, (iii) gap extend: 0.5, (iv) output format: pair, (v) end gap penalty: false, (vi) end gap open: 10, (vii) end gap extend: 0.5.

The aptamer of the invention typically comprises from 20 to 150 nucleotides in length, preferably from 30 to 100 nucleotides in length, for instance from 25 to 90 nucleotides in length, from 30 to 80 nucleotides in length or from 30 to 60 nucleotides in length. Accordingly, the aptamer of the invention may have 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 in length.

In a particular embodiment, the aptamer of the invention comprises a polynucleotide which differs from a polynucleotide selected from the group of SEQ ID No 66 and SEQ ID NO:67 in virtue of 1 to 15 nucleotide modifications, preferably in virtue of 1 to 10 nucleotide modifications such as 1, 2, 3, 4, 5, 6, 7, 8, 9, or 10 nucleotide modifications.

As used herein, a “nucleotide modification” refers to the deletion of a nucleotide, the insertion of a nucleotide, or the substitution of a nucleotide by another nucleotide as compared to the reference sequence.

The aptamers of the invention may also comprise primers at its 3′- and 5′-terminus useful for its amplification by PCR. In some embodiments, these primer sequences can be included or partially included in the core sequence and thus participate in binding interactions with fibrinogen. In some other embodiments, these primer sequences are outside the core sequence and may not play any role in the interaction of the aptamer with fibrinogen. In some further embodiments, the aptamer is devoid of primer sequences.

In some alternate or additional embodiments, the aptamer of the invention may comprise a polynucleotide of 2 to 40 nucleotides in length linked to the 5′-end and/or the 3′-end of the core sequence.

In a certain aspect, the invention relates to an aptamer which specifically binds to fibrinogen and which is of formula (I)

5′-[NUC1]ₘ-[CENTRAL]-[NUC2]ₙ-3′ (I)

### Wherein

n and m are integers independently selected from 0 and 1,
[NUC1] is a polynucleotide comprising from 2 to 40 nucleotides, preferably from 15 to 25 nucleotides
[NUC2] is a polynucleotide comprising from 2 to 40 nucleotides, preferably from 15 to 25 nucleotides and
[CENTRAL] is a polynucleotide having at least 70% of sequence identity with a nucleotide sequence selected from the group consisting of SEQ ID NO:68, SEQ ID NO:69, SEQ ID NO:70, SEQ ID NO:71, SEQ ID NO:72, SEQ ID NO:73, SEQ ID NO:74, SEQ ID NO:94 and SEQ ID NO:95.

When n=m=0, [NUC1] and [NUC2] are absent and the aptamer consists of the sequence [CENTRAL]. When n=0 and m=1, [NUC2] is absent and [NUC1] is present, the aptamer is thus of formula (Ia):

5′-[NUC1]-[CENTRAL]-3′.

When n=1 and m=0, [NUC1] is absent and [NUC2] is present, the aptamer is thus of formula (Ib):

5′-[CENTRAL]-[NUC2]-3′.

In some embodiments, [NUC1] comprises, or consists of, a polynucleotide of SEQ ID No 75 or a polynucleotide which differs from SEQ ID No 75 in virtue of 1, 2, 3, or 4 nucleotide modifications. In some other or additional embodiments, [NUC2] comprises, or consists of, a polynucleotide of SEQ ID No 76 or a polynucleotide which differs from SEQ ID No 76 in virtue of 1, 2, 3, or 4 nucleotide modifications.

In another aspect, the invention relates to an aptamer directed against fibrinogen and which has at least 70%, such as at least 75%, 80%, 85%, 90%, 95%, 96%, 97% or 98% of sequence identity with a nucleotide sequence selected from the group consisting of SEQ ID NO:1 to SEQ ID NO:67. For instance, the aptamer of the invention may have at least 70% of sequence identity with a nucleotide sequence selected from SEQ ID NO:1, SEQ ID NO:2, SEQ ID NO:58, SEQ ID NO:60, SEQ ID NO:65, SEQ ID NO:66 and SEQ ID NO:67.

The Applicant performed an extensive analysis of the sequences and the possible conformations of the aptamers as defined above. This analysis led to the identification of two subgroups of aptamers, each subgroup being characterized by specific structural and functional properties.

First Subgroup of Aptamers According to the Invention

The first subgroup of aptamers encompasses aptamers directed against fibrinogen which comprises a core sequence displaying a high sequence identity with the core sequence of SEQ ID No 66. This first subgroup encompass aptamers of SEQ ID NO:1-57 and the aptamer consisting of the core sequence of SEQ ID NO:66.

Accordingly, the invention also relates to an aptamer which selectively binds to fibrinogen and which comprises a polynucleotide having at least 70%, 75%, 80%, 85%, 90%, 92%, 94%, 95%, 96%, 97% or 98% of sequence identity with SEQ ID No 66. Preferably, said aptamer has from 20 to 110 nucleotides in length, in particular from 25 to 100 nucleotides in length, such as 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 in length. In particular, the aptamer can have from 35 to 65 nucleotides in length.

In some embodiments, the aptamer comprises a polynucleotide of SEQ ID No 66, or a polynucleotide having a nucleotide sequence which differs from SEQ ID NO:66 in virtue of 1 to 16 nucleotide modifications, such as 1, 2, 3, 4, 5, 6, 7, 8, 9, 10, 11, 12, 13, 14, or 15 nucleotide modifications. As mentioned above, the nucleotide modifications(s) can be of any type. A nucleotide modification may be a deletion of one nucleotide, the insertion of one nucleotide or the substitution/replacement of one nucleotide by another nucleotide.

The alignment of the core sequence of SEQ ID NO:66 with aptamers of SEQ ID NO:1-57 showed that certain nucleotides are not conserved among the aptamers belonging to the first subgroup. Said positions encompass positions 19, 20, 21, 24, 27, 32, 33, 35, 42, 45, 46, 47, 50, 54, 55 and 57, the numbering referring to nucleotide numbering in SEQ ID NO:66.

Other modifications, in particular one or several deletions, can be introduced at positions 1 and 2 of SEQ ID NO:66 but also in the third stem of the core sequence of SEQ ID NO:66, as shown in FIG. 1A. In particular, the Applicant introduced from one to six deletions in the third stem of the aptamer of SEQ ID NO:66, in particular at positions 14-22, without any significant loss of affinity to fibrinogen as compared to the parent core sequence of SEQ ID NO:66.

Accordingly, the nucleotide modification(s) as compared to SEQ ID NO:66 may be present at one or several of these nucleotide positions. In some embodiments, the aptamer of the invention comprises a polynucleotide which differs from SEQ ID NO:66 in virtue of 1 to 20, preferably from 1 to 14, in particular from 1, 2, 3, 4, 5, or 6 nucleotide modifications at nucleotide positions selected from 1, 2, 11-25, 32-35, 42, 45-47, 50 and 54-58, preferably at nucleotide positions selected from 1, 2, 14-22, 24, 27, 32, 33, 35, 42, 45, 46, 47, 50, 54, 55 and 57, the numbering referring to nucleotide numbering in SEQ ID NO:66. Preferably, the nucleotide modification(s) is/are nucleotide replacement(s) or deletion(s).

In some particular embodiments, said nucleotide modification(s) occur(s) at nucleotide positions selected from 20, 35, 42, and 55. In some particular embodiments, the aptamer of the invention may comprise a polynucleotide which differ from SEQ ID NO:66 in virtue of at most 4 nucleotide modifications which preferably occur at positions selected from 20, 35, 42, and 55, the numbering referring to nucleotide numbering in SEQ ID NO:66.

For instance, the aptamer of the invention may comprise a polynucleotide of SEQ ID NO:66, or a polynucleotide having a nucleotide sequence which differs from SEQ ID NO:66 in virtue of 1, 2, or 3 nucleotide modification(s), preferably in virtue of 1, 2 or 3 nucleotide substitutions(s), said nucleotide modification(s) being at nucleotide position(s) selected from the group consisting of 19, 20, 21, 24, 27, 32, 33, 35, 42, 45, 46, 47, 50, 54, 55 and 57, the numbering referring to nucleotide numbering in SEQ ID NO:66.

In some other embodiments, the aptamer of the invention may comprise the polynucleotide of SEQ ID NO:66, or a polynucleotide having a nucleotide sequence which differs from SEQ ID NO:66 in virtue of 1-14 nucleotide deletion(s), preferably in virtue of 1, 2, 3, 4, 5, 6 or 7 nucleotide deletion(s), said nucleotide deletion(s) being at nucleotide position(s) selected from the group consisting of 1, 2, 14, 15, 16, 17, 18, 19, 20, 21 and 22, the numbering referring to nucleotide numbering in SEQ ID NO:66.

In some additional embodiments, the aptamer of the invention may comprise a polynucleotide of SEQ ID NO:66, or a polynucleotide having nucleotide sequence which differs from SEQ ID NO:66 in virtue of one of the following nucleotide deletion combinations:
nucleotide deletions at positions 19, 20, and 21,
nucleotide deletions at positions 18, 19, 20 and 21
nucleotide deletions at positions 15, 16, 19 and 20,
nucleotide deletions at positions 14, 15, 16, 20 and 21, and
nucleotide deletions at positions 14, 15, 16, 20, 21 and 22.
   the numbering referring to nucleotide numbering in SEQ ID NO:66.

Alternatively or additionally, nucleotide deletions can be present at positions 1 and 2 the numbering referring to the nucleotide numbering in SEQ ID NO:66.

As shown in Example 5, the Applicant identified variants of SEQ ID NO:66 able to bind to fibrinogen in a competitive manner.

In some additional or alternate embodiments, the aptamer of the invention is an aptamer which selectively binds to fibrinogen and which comprises a polynucleotide selected from the group consisting of SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, and SEQ ID NO:93 or having a nucleotide sequence which differs in virtue of 1, 2, 3, 4 or 5 nucleotide modifications from a sequence selected from the group consisting of SEQ ID NO:80-93.

Preferred variants of SEQ ID NO:66 encompass aptamers of SEQ ID NO:80-87.

The first subgroup of aptamers according to the invention also encompass aptamers directed against fibrinogen and which comprises a polynucleotide having at least 80%, 85%, 90%, 92%, 94%, 95%, 96%, 97% or 98% of sequence identity with SEQ ID NO:1.

In particular, the aptamer may be of SEQ ID NO:1 or may have a nucleotide sequence which differs from SEQ ID NO:1 in virtue of 1-20, in particular 1, 2, 3, 4, 5, 6, 7, 8, 9 or 10 nucleotide modifications. Said nucleotide modification(s) may be preferably present at position(s) as described above for SEQ ID NO:66.

As explained in the below section entitled “Method for obtaining aptamers of the invention”, certain aptamers of the invention have been identified from a candidate mixture consists of a multitude of single-stranded DNAs (ssDNA), wherein each ssDNA comprises a central random sequence of about 20 to 100 nucleotides flanked by specific sequences of about 15 to 40 nucleotides which function as primers for PCR amplification.

In some alternate or additional embodiments, the aptamer of the invention is of formula (I) wherein:

5′-[NUC1]ₘ-[CENTRAL]-[NUC2]ₙ-3′ (I)

### Wherein:

[CENTRAL] is a polynucleotide having at least 70%, preferably at least 80%, for instance at least 85%, 90%, 93%, 95%, 96%, 97%, 98%, 99% or 100% of sequence identity with

SEQ ID No 94,
n and m are integers independently selected from 0 and 1,
[NUC1] is a polynucleotide comprising from 2 to 40 nucleotides, preferably from 15 to 25 nucleotides, and
[NUC2] is a polynucleotide comprising from 2 to 40 nucleotides, preferably from 15 to 25 nucleotides

In a preferred embodiment, m=1 and [NUC1] comprises, or consists of, a polynucleotide of SEQ ID NO:75 or which differs from SEQ ID NO:75 in virtue of 1, 2, 3, or 4 nucleotide modifications.

An example of aptamer of formula (I) is the aptamer of SEQ ID NO:66.

Accordingly, the aptamer of the invention is of the following formula:

5′-[NUC1]-[CENTRAL]-[NUC2]ₙ-3′

wherein n is 0 or 1.

In some further embodiments, [NUC2] may comprise, or consist of, a polynucleotide of SEQ ID NO:76 or a polynucleotide which differs from SEQ ID No 76 in virtue of 1, 2, 3, or 4 nucleotide modifications.

In a specific aspect, the aptamer of the invention may be an aptamer of formula (I)

5′-[NUC1]ₘ-[CENTRAL]-[NUC2]ₙ-3′ (I)

wherein:
m is 1,
n is 0 or 1,
[NUC1] is a polynucleotide of SEQ ID NO:75 or which differs from SEQ ID NO:75 in virtue of 1, 2, 3, or 4 nucleotide modifications, preferably nucleotide deletions.
[NUC2] is a polynucleotide having from 2 to 40 nucleotides in length, and
[CENTRAL] is the polynucleotide of SEQ ID NO:94, or has a nucleotide sequence which differs from SEQ ID NO:94 in virtue of 1, 2, 3, 4, 5, or 6 nucleotide modifications, preferably nucleotide deletions.

In some embodiments, [NUC2] is a polynucleotide of SEQ ID NO:76, or has a sequence which differs from SEQ ID NO:76 in virtue of 1, 2, 3 or 4 nucleotide modifications, said nucleotide modifications being preferably selected from nucleotide substitutions and/or nucleotide deletions.

In some embodiments, the aptamers of said first subgroup may comprise a conformation moiety as shown in FIG. 1A by the highlighted nucleotides. In a more general aspect, the aptamers of the invention may have a nucleotide sequence comprising nucleotide domains able to form a conformation moiety comprising a central loop comprising from 15 to 19 nucleotides preferably 17 nucleotides bearing:
a first stem having a length of 4 to 6 nucleotides, preferably 5 nucleotides,
a second stem having from 2 to 4, preferably 3 nucleotides connected to a loop comprising from 13 to 15, preferably 14 nucleotides, and
optionally a third stem having from 2 to 8, preferably 7 nucleotides connected to a loop comprising from 2 to 4, preferably 3 nucleotides.

In some preferred embodiments, the first stem is adjacent to the second stem and separated by 2 nucleotides from the third stem.

The aptamers belonging to the first subgroup of the invention may be able to bind to fibrinogen at a slightly acidic pH as defined above, preferably at a pH of around 6.3. In particular, the aptamers of the first group may bind to fibrinogen in a pH-dependent manner.

In some embodiments, said aptamers display an increased affinity for fibrinogen at pH 6.3 as compared to a slight basic pH such as pH 7.4. In some embodiments, said aptamer does not bind to fibrinogen at a pH of 7.0 or above

Said subgroup of aptamers may be also able to bind to fibrinogen in the presence of Mg²⁺ In some embodiments, said aptamers may display a binding affinity for fibrinogen which depends on the pH and/or the presence of Mg²⁺ in the medium. For instance, the binding affinity of the aptamer for the fibrinogen may be increased in the presence of Mg²⁺ at a concentration in the mM range, for instance from 1 to 10 mM, as compared to the same medium devoid of Mg²⁺. As a further example, the aptamer of the invention specifically bind to fibrinogen at a pH of about 6.3, and does not bind to fibrinogen at pH above 7.0, such as pH 7.4.

Such properties are for instance illustrated herein for the aptamer of SEQ ID NO:66 in the below section entitled “Examples”.

Second Subgroup of Aptamers According to the Invention

The second subgroup of aptamers encompass, without being limited to, aptamers of SEQ ID NO:58-65 and the core sequence of SEQ ID NO:67.

Aptamers of SEQ ID NO:58-65 and SEQ ID NO:67 comprise two consensus moieties in their sequences, namely GTTGGTAGGG (SEQ ID NO:77) which is upstream of GGTGTAT (SEQ ID NO:78) as shown in the sequence alignment of FIG. 1C. Without to be bound by any theory, the Applicant believes that these consensus moieties are located in a region of the aptamers which forms a loop as evidenced in FIG. 1B for the aptamer of SEQ ID NO:58. This conformational moiety may play a role in the binding of said aptamers to fibrinogen. Accordingly, this second subgroup of aptamers encompasses aptamers which specifically bind to fibrinogen and which comprise the nucleotide moieties of SEQ ID NO:77 and SEQ ID NO:78, SEQ ID NO:77 being upstream of SEQ ID NO:78 in the core sequence of said aptamer. Preferably, said aptamer comprises a nucleotide moiety of formula (III)

5′-[SEQ ID NO: 79]-[X1]-[SEQ ID NO: 77]-[X2]- SEQ
ID NO: 78]-3′

wherein:
[X2] and [X1] independently denote a nucleotide or an oligonucleotide of 2 to 5 nucleotides in length, preferably of 2 or 3 nucleotides in length,
[SEQ ID NO:77] is an oligonucleotide of SEQ ID NO:77 (namely GTTGGTAGGG),
[SEQ ID NO:78] is an oligonucleotide of SEQ ID NO:78 (namely GGTGTAT) and
[SEQ ID NO:79] is an oligonucleotide of SEQ ID NO:79 (namely TGT)

For instance, the aptamer of the invention may comprise a moiety of formula (III) wherein X1 denotes one nucleotide, e.g. G or T, and X2 is an oligonucleotide of 3 nucleotides in length. In some embodiments, the aptamers may further comprise a polynucleotide having at least 70%, 75%, 80%, 85%, 90% or 95% of sequence identity with a sequence selected from the group consisting of SEQ ID NO:67, SEQ ID NO:68, SEQ ID NO:69, SEQ ID NO:70, SEQ ID NO:71, SEQ ID NO:72, SEQ ID NO:73, SEQ ID NO:74 and SEQ ID NO:95.

Accordingly, the second subgroup of aptamers encompasses aptamers which specifically bind to fibrinogen and which comprise a polynucleotide:
having at least 70%, 75%, 80%, 85%, 90% or 95% of sequence identity with a nucleotide sequence selected from the group consisting of SEQ ID NO:67, SEQ ID NO:68, SEQ ID NO:69, SEQ ID NO:70, SEQ ID NO:71, SEQ ID NO:72, SEQ ID NO:73, SEQ ID NO:74 and SEQ ID NO:95, and
comprising a nucleotide moiety of formula (III)

5′-[SEQ ID NO: 79]-[X1]-[SEQ ID NO: 77]-[X2]- SEQ
ID NO: 78]-3′

Wherein:
[X2] and [X1] independently denote a nucleotide or an oligonucleotide of 2 to 5 nucleotides in length, preferably of 2 or 3 nucleotides in length,
[SEQ ID NO:77] is an oligonucleotide of SEQ ID NO:77 (namely GTTGGTAGGG),
[SEQ ID NO:78] is an oligonucleotide of SEQ ID NO:78 (namely GGTGTAT) and
[SEQ ID NO:79] is an oligonucleotide of SEQ ID NO:79 (namely TGT)

The aptamer of the invention has preferably from 20 to 150 nucleotides in length, in particular from 25 to 100 nucleotides in length, such as 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 in length.

In an additional or alternate embodiment, the aptamer of the invention specifically binds to fibrinogen and comprises a polynucleotide having at least 70%, 75%, 80%, 85%, 90% or 95% of sequence identity with the core sequence of SEQ ID NO:67.

In some embodiments, the aptamer of the invention specifically binds to fibrinogen and comprise a polynucleotide of SEQ ID NO:67, or which differs from SEQ ID NO:67 in virtue of 1, 2, 3, 4, 5, or 6 nucleotide modifications.
In a particular aspect, the aptamer of the invention specifically binds to fibrinogen and is of formula (I): 5′-[NUC1]ₘ-[CENTRAL]-[NUC2]ₙ-3′ Wherein: n and m are integers independently selected from 0 and 1,
[NUC1] is a polynucleotide comprising from 2 to 40 nucleotides, preferably from 15 to 25 nucleotides[NUC2] is a polynucleotide comprising from 2 to 40 nucleotides, preferably from 15 to 25 nucleotides and
[CENTRAL] is a polynucleotide having at least 70%, 75%, 80%, 85%, 90% or 95% of sequence identity with a nucleotide sequence selected from the group consisting of SEQ ID NO:68, SEQ ID NO:69, SEQ ID NO:70, SEQ ID NO:71, SEQ ID NO:72, SEQ ID NO:73 SEQ ID NO:74 and SEQ ID NO:95 and which comprises a nucleotide moiety of formula (III) as defined above.

In some embodiments, the aptamer of the invention is an aptamer of formula (I) comprising at least 1, 2, 3 or all following features:
n=m=1.
[NUC1] comprises, or consists of, a polynucleotide of SEQ ID No 75 or a polynucleotide which differs from SEQ ID No 75 in virtue of 1, 2, 3, or 4 nucleotide modifications,
[NUC2] comprises, or consists of, a polynucleotide of SEQ ID No 76 or a polynucleotide which differs from SEQ ID No 76 in virtue of 1, 2, 3, or 4 nucleotide modifications,
[CENTRAL] is a polynucleotide having at least 80%, preferably at least 85% of sequence identity with SEQ ID NO:95 and which comprises a nucleotide moiety of formula (III) as defined above

In another alternate or particular aspect, the aptamer of the invention specifically binds to fibrinogen and comprise a polynucleotide having at least 70%, 75%, 80%, 85%, 90% or 95% of sequence identity with a polynucleotide selected from SEQ ID NO:58, SEQ ID NO:59, SEQ ID NO:60, SEQ ID NO:61, SEQ ID NO:62, SEQ ID NO:63, SEQ ID NO:64, SEQ ID NO:65 and SEQ ID NO:67.

In some embodiments, the aptamers of said second subgroup may comprise a conformation moiety as shown in FIG. 1B by the highlighted nucleotides. In a more general aspect, the aptamers of the invention may have a nucleotide sequence comprising nucleotide domains able to form a conformation moiety comprising a stem having from 2 to 5 nucleotides in length, for instance 4 nucleotides linked to a loop of 23 to 27 nucleotides. Preferably the loop is devoid of any supplementary stem-loop moiety and/or stem moiety.

The aptamers belonging to said second subgroup may be able to bind to fibrinogen at a slightly acidic pH, preferably at a pH of around 6.3. In some embodiments, said aptamers display an increased binding to fibrinogen at pH 6.3 as compared to pH above 7.0 such as pH 7.4.

Preferably, said aptamers do not bind to fibrinogen at a pH above 7.0. More generally, the aptamers of the second group may bind to fibrinogen in a pH-dependent manner.

This subgroup of aptamers may be also able to bind to fibrinogen in the absence of Mg²⁺. In some embodiments, said aptamers may display a binding affinity for fibrinogen which depends on the pH and/or the presence of Mg²⁺ in the medium. For instance, the binding affinity of the aptamer for the fibrinogen may be decreased in the presence of Mg²⁺, e.g. in a medium comprising Mg²⁺ in the mM range as compared to the same medium devoid of Mg²⁺

Affinity Ligands and Affinity Supports of the Invention

The invention also relates to affinity ligands comprising an aptamer directed against fibrinogen. Said affinity ligands may be immobilized onto a solid support for the detection, the quantification, or the purification of fibrinogen. Alternatively or additionally, the affinity ligand may comprise a mean for detection. A mean of detection may be any compound generating a signal quantifiable, preferably by instrumented reading. Suitable detectable labels may be selected, for example, from the group consisting of colloidal metals such as gold or silver; non-metallic colloids such as colloidal selenium, tellurium or sulphur particles; fluorescent, luminescent and chemiluminescent dyes, fluorescent proteins such as GFP, magnetic particles, radioactive elements, and enzymes such as horseradish peroxidase

Typically, the affinity ligand of the invention comprises (i) an aptamer moiety, i.e. an aptamer directed against fibrinogen as defined above linked to at least one (ii) non-aptamer entity useful for immobilization on an appropriate substrate. Preferably, the non-entity aptamer is preferably linked to the 5′- or the 3′-end of the aptamer.

In certain embodiment, the affinity ligand may comprise a mean of immobilization linked to the aptamer moiety directly or by a spacer group. Accordingly, the affinity ligand may comprise, or consist of, a compound of formula (IV):

[IMM]-([SPACER])ₚ-[APTAMER] wherein
[APTAMER] denotes an aptamer as defined above,
[SPACER] is a spacer group,
[IMM] is a moiety for the immobilization of the aptamer onto a support and
p is 0 or 1.

p is 0 means that the spacer is absent and that [IMM] is directly linked to [APTAMER], preferably at the 3′ or the 5′-end of aptamer.

p is 1 means that the spacer is present and links to [IMM] and [APTAMER].

The spacer group is typically selected to decrease the steric hindrance of the aptamer moiety and improve its accessibility while preserving the aptamer capability of specifically binding to fibrinogen. The spacer group may be of any type. The spacer may be a non-specific single-stranded nucleotide, i.e. which is not able to bind to a protein, including fibrinogen. Typically the spacer may comprise from 2 to 20 nucleotides in length. Examples of appropriate nucleic spacers are polyA and polyT. In some other embodiments, the spacer may be a non-nucleic chemical entity. For instance, the spacer may be selected from the group consisting of a peptide, a polypeptide, an oligo- or polysaccharide, a hydrocarbon chain optionally interrupted by one or several heteroatoms and optionally substituted by one or several substituents such as hydroxyl, halogens, or C₁-C₃ alkyl; polymers including homopolymers, copolymers and block polymers, and combinations thereof. For instance the spacer may be selected from the group consisting of polyethers such as polyethylene glycol (PEG) or polypropylene glycol, polyvinylic alcool, polyacrylate, polymethacrylate, polysilicone, and combination thereof. For instance, the spacer may comprise several hydrocarbon chains, oligomers or polymers linked by any appropriate group, such as a heteroatom, preferably −0- or −5-, —NHC(O)—, —OC(O)—, —NH—, —NH—CO—NH—, —O—CO—NH—, phosphodiester or phosphorothioate. Such spacer chains may comprise from de 2 á 200 carbon atoms, such as from 5 to 50 carbon atoms. Preferably, the spacer is selected from non-specific oligonucleotides, hydrocarbon chains, polyethers, in particular polyethelene glycol and combinations thereof.

For instance, the spacer comprises at least one polyethylene glycol moiety comprising from 2 to 20 monomers. For instance, the spacer may comprise from 1 to 4 triethyleneglycol blocks linked together by appropriate linkers. For example, the spacer may be a C12 hydrophilic triethylene glycol ethylamine derivative. Alternatively, the spacer may be a C₂-C₂₀ hydrocarbon chain, in particular a C₂-C₂₀ alkyl chain such as a C12 methylene chain.

The spacer is preferably link to the 3′-end or the 5-end of the aptamer moiety.

[IMM] refers to any suitable moiety enabling to immobilize the affinity ligand onto a substrate, preferably a solid support. [IMM] depends on the type of interactions sought to immobilize the affinity ligand on the substrate.

For instance, the affinity ligand may be immobilized thanks to specific non-covalent interactions including hydrogen bonds electrostatic forces or Van der Waals forces. For example, the immobilization of the affinity ligand onto the support may rely ligand/anti-ligand couples (e.g. antibody/antigen such as biotin/anti-biotin antibody and digoxygenine/anti-digoxigenin antibody, or ligand/receptor) and protein binding tags. A multitude of protein tags are well-known by the skilled person and include, for example, biotin (for binding to streptavidin or avidin derivatives), glutathione (for binding to proteins or other substances linked to glutathione-S-transferase), maltose (for binding to proteins or other substances linked to maltose binding protein), lectins (for binding to sugar moieties), c-myc tag, hemaglutinin antigen (HA) tag, thioredoxin tag, FLAG tag, polyArg tag, polyHis tag, Strep-tag, chitin-binding domain, cellulose-binding domain, and the like. In some embodiments, [IMM] denotes biotin. Accordingly, the affinity ligand of the invention is suitable to be immobilized on supports grafted with avidin or streptavidin.

Alternatively, the affinity ligand may be suitable for covalent grafting on a solid support. [IMM] may thus refer to a chemical entity comprising a reactive chemical group. The chemical entity has typically a molecular weight below than 1000 g·mol⁻¹, preferably less than 800 g·mol⁻¹ such as less than 700, 600, 500 or 400 g·mol⁻¹. The reactive groups can be of any type and encompasses primary amine, maleimide group, sulfhydryl group and the likes.

For instance, the chemical entity may derive from SIAB compound, SMCC compound or derivatives thereof. The use of sulfo-SIAB to immobilize oligonucleotides is for instance described in Allerson et al., RNA, 2003, 9:364-374

In some embodiments, [IMM] comprises a primary amino group. For instance, [IMM] may be —NH₂ or a C₁₋₃₀ aminoalkyl preferably a C₁-C₆ aminoalkyl. An affinity ligand wherein [IMM] comprises a primary suitable group is suitable for immobilization on support having thereon activated carboxylic acid groups. Activated carboxylic acid groups encompass, without being limited to, acid chloride, mixed anhydride and ester groups. A preferred activated carboxylic acid group is N-hydroxysuccinimide ester.

As mentioned above, [IMM]-([SPACER])p is preferably links to the 3′-end or the 5′ end of the aptamer. The terminus of the aptamer moiety which is not linked to [IMM]-([SPACER])p may comprise a chemically modified nucleotide such as 2′-o-methyl or 2′ fluoropyrimidine, 2′-ribopurine, phosphoramidite, an inverted nucleotide or a chemical group such as PEG or cholesterol. Such modifications may prevent the degradation, in particular the enzymatic degradation of the ligands. In other embodiments, said free terminus of the aptamer (i.e. which is not bound to [IMM] or to [SPACER]) may be linked to a mean of detection as described above.

A further object of the invention is an affinity support capable of selectively binding fibrinogen, which comprises thereon a plurality of affinity ligands as defined above.

The affinity ligands can be immobilized onto the solid support by non-covalent interactions or by a covalent bond(s).

In some embodiments, the affinity ligands are covalently grafted on said support. Typically, the grafting is performed by reacting the chemical reactive group present in the moiety [IMM] of the ligand with a chemical reactive group present on the surface of the solid support.

Preferably, the chemical reactive group of the ligand is a primary amine group and that present on the solid support is an activated carboxylic acid group such as a NHS-activated carboxylic group (namely N-hydroxysuccimidyle ester). In this case, the grafting reaction can be performed at a pH lower than 6, for instance at a pH from 3.5 to 4.5 as illustrated in Example 2 and described in WO2012090183, the disclosure of which being incorporated herein by reference.

The solid support of the affinity support may be of any type and is selected depending on the contemplated use.

For instance, the solid support may be selected among plastic, metal, and inorganic support such as glass, nickel/nickel oxide, titanium, zirconia, silicon, strained silicon, polycrystalline silicon, silicon dioxide, or ceramic. The said support may be contained in a device such as microelectronic device, microfluidic device, a captor, a biosensor or a chip for instance suitable for use in SPR. Alternatively, the support may be in the form of beads, such as polymeric, metallic or magnetic beads. Such supports may be suitable for detection and diagnostic purposes.

In other embodiments, the solid support may be a polymeric gel, filter or membrane. In particular, the solid support may be composed of agarose, cross-linked agarose, cellulose or synthetic polymers such as polyacrylamide, polyethylene, polyamide, polysulfone, and derivatives thereof. Such supports may be suitable for the purification of fibrinogen. For instance, the solid support may be a support for chromatography, in particular for liquid affinity chromatography. For instance, the affinity support of the invention may be appropriate for carrying out affinity chromatography at the industrial scale. The affinity support of the invention may thus be used as stationary phase in chromatography process, for instance, in column chromatography process or in batch chromatography process.

Uses of the aptamers and affinity ligands according to the invention in the purification of fibrinogen and in other fields

In an additional aspect, the aptamers and the affinity ligands of the invention may be used in the diagnostic and detection field. In particular, the aptamers and the affinity ligands of the invention may be useful for the diagnostic or the prognostic of diseases or disorders associated with a variation of fibrinogen plasmatic level.

For instance, the aptamers or the ligands of the invention may be used in the diagnostic or the prognostic of disorders such as fibrinogen deficiencies. The aptamers or the ligands of the invention may be used in the diagnostic or the prognostic of disorders wherein the plasma level of fibrinogen is a biomarker of the occurrence or the outcome of the disorders.

In another aspect, the aptamers may be also used in the treatments of coagulation disorders.

In another aspect, the invention relates to a method for capturing fibrinogen, said method comprising:
providing a solid support having an aptamer or an affinity ligand of the invention immobilized thereon,
contacting said solid support with a solution containing fibrinogen, whereby fibrinogen is captured by the formation of a complex between fibrinogen and said aptamer or said affinity ligand immobilized on the solid support.

In some embodiments, the method may comprise one or several additional steps step such as:
a step of releasing fibrinogen from said complex,
a step of recovering fibrinogen from said complex
a step of detecting the formation of the complex between fibrinogen and said aptamer or affinity ligand
a step of quantifying fibrinogen,

The detection of the complex and the quantification of fibrinogen (or that of the complex) may be performed by any method known by the skilled artisan. For instance, the detection and the quantification may be performed by SPR as illustrated in the Examples.

Alternatively, one may use an ELISA-type assay wherein a labelled anti-fibrinogen antibody is used for detecting or quantifying the complex formed between fibrinogen and the affinity ligands of the invention. The anti-fibrinogen antibody may be labelled with a fluorophore or coupled to an enzyme suitable for the detection, such as the horseradish peroxidase.

The invention also relates to a complex comprising (i) fibrinogen and (ii) an aptamer or an affinity ligand directed to fibrinogen, as described above.

As fully illustrated in Example 3 and 4 below, the aptamers of the invention are particularly suitable for a use in the purification of fibrinogen.

In a particular embodiment, the invention also relates to the use of an aptamer, an affinity ligand or an affinity support of the invention for the purification of fibrinogen. A further object of the invention is thus a method for purifying fibrinogen from a starting composition comprising:
a. contacting said starting composition with an affinity support as defined above, in conditions suitable to form a complex between (i) the aptamers or the affinity ligands immobilized on said support and (ii) fibrinogen,
b. releasing fibrinogen from said complex, and
c. recovering fibrinogen in purified form.

A further object of the invention is a method for preparing a purified fibrinogen composition from a starting fibrinogen-containing composition comprising:
a. contacting said starting composition with an affinity support as defined above, in conditions suitable to form a complex between (i) the aptamers or the affinity ligands immobilized on said support and (ii) fibrinogen
b. releasing fibrinogen from said complex, and
c. recovering a purified fibrinogen composition.

As used herein, the starting composition may be any composition which potentially comprises fibrinogen. The starting composition may comprise contaminants from which fibrinogen is to be separated.

The contaminants may be of any type and depend on the nature of the starting composition. The contaminants encompass proteins, salts, hormones, vitamins, nutriments, lipids, cell debris such as cell membrane fragments and the like. In some embodiments, the contaminants may comprise blood proteins such as clotting factors, fibronectin, albumin, immunoglobulin, plasminogen alpha-2-macroglobulin and the like.

In some other embodiments, the contaminant may comprise non-human proteins, in particular non-human proteins endogenously expressed by a recombinant host such as a recombinant cell, bacteria or yeast, or a transgenic animal.

Typically, the starting composition may be, or may derive from, a cell culture, a fermentation broth, a cell lysate, a tissue, an organ, or a body fluid. As used herein, a “starting composition” derives from an entity of interest, such as milk, blood or cell culture, means that the starting composition is obtained from said entity by subjecting said entity to one or several treatment steps. For instance, the entity of interest may be subjected to one or several treatments such as cell lysis, a precipitation step such as salt precipitation, cryo-precipitation or flocculation, a filtration step such as depth filtration or ultrafiltration, centrifugation, clarification, chromatography, an extraction step such as a liquid-liquid or a solid-liquid extraction, viral inactivation, pasteurization, freezing/thawing steps and the like. For instance, a starting composition derived from blood encompass, without being limited to, plasma, a plasma fraction and a blood cryoprecipitate.

In some embodiments, the starting solution derived from blood, preferably from human blood. The starting composition may be selected from plasma, plasma fraction, for instance Fraction I obtained by Cohn's ethanol fractionation process, and blood cryoprecipitate. In some embodiments, the starting composition is an immunoglobulin-depleted plasma fraction and/or an albumin-depleted plasma fraction and/or a vitamin K-dependent coagulation protein-depleted blood or plasma fraction.

In some other embodiments, the starting composition is obtained from a recombinant host. Preferably, the recombinant host is a transgenic animal, such as a non-human transgenic mammal. The transgenic non-human mammal may be any animal which has been genetically modified so as to express human fibrinogen. Preferably, the human fibrinogen is expressed in a body fluid of said transgenic animal.

The starting solution may thus be, or may derive from, a body fluid of a transgenic animal. Body fluids encompass, without being limited to, blood, breast milk, and urine.

In a particular embodiment, the starting composition is, or derives from, milk from a transgenic non-human mammal. Methods for producing a transgenic animal able to secrete a protein of interest in milk are well-known in the state of art. Typically, such methods encompass introducing a genetic construct comprising a gene coding for the protein of interest operably linked to a promoter from a protein which is naturally secreted in milk (such as casein promoter or WHAP promoter) in an embryo of a non-human mammal. The embryo is then transferring in the uterus of a female from the same animal species and which has been hormonally prepared for pregnancy.

In some preferred embodiments, the starting composition may be selected from human blood, transgenic milk and derivatives thereof.

The affinity support used in the methods of the invention may be any affinity supports described hereabove. Preferably, the affinity support is an affinity support for performing affinity chromatography. Indeed, the methods for purifying fibrinogen or preparing a purified composition of fibrinogen are preferably based on chromatography technologies, for instance in batch or column modes, wherein the affinity support plays the role of the stationary phase. In step a), an appropriate volume of the starting composition containing fibrinogen is contacting with an a affinity support in conditions suitable to promote the specific interactions of the anti-fibrinogen aptamer moieties present on the surface of the affinity support with the fibrinogen, whereby a complex is formed between fibrinogen molecules and said aptamer moieties. In step a), Fibrinogen is thus retained on the affinity support. The binding between the aptamer moieties and fibrinogen molecules may be enhanced by performing step a) at a slightly acidic pH. In some embodiments, step a) is performed at a pH lower than 7.0, preferably lower than 6.9, 6.8, or 6.7. In particular step a) may be performed at a pH from 6.0 to 6.8, preferably at a pH of 6.0 to 6.5, such as 6.0, 6.1, 6.2, 6.3, 6.4, or 6.5. For instance, step a) may be performed at a pH of 6.1 to 6.5 such as a pH of 6.3. In a more general aspect, the pH condition of step a) may be selected so as to promote the binding of fibrinogen onto the affinity support while minimizing the binding of the other molecules onto the affinity support.

Typically, step a) is performed in the presence of a buffer solution (called hereafter a “binding buffer”). The binding buffer can be mixed with the starting composition prior to step a) or can be added during step a). The binding buffer is typically an aqueous solution containing a buffer agent. The buffer agent may be selected so as to be compatible with fibrinogen and the affinity support and so as to obtain the desired pH for step a). For instance, for obtaining a pH of about 6.3 the buffer agent may be selected from, without being limited to, 3-(N-morpholino)propanesulfonic acid (MOPS), 2-(N-morpholino)ethanesulfonic acid (MES), HEPES, Bis-TRIS citrate and acetate. The buffering agent may be present at a concentration of about 5 mM to 1 M, for instance from 10 mM to 500 mM, for instance from 10 mM to 200 mM such as about 50 mM.

Without to be bound by any theory, the presence of salts may promote the formation of the complex between fibrinogen and the aptamer moieties of the solid support and/or prevent the binding of the other molecules present in the starting composition. Typically, step a) may be performed in the presence of sodium chloride, for instance at a concentration ranging from 10 mM to 500 mM, preferably from 50 mM to 350 mM, or from 100 mM to 200 mM such as about 150 mM.

The presence of divalent cations may modulate the binding of fibrinogen to the aptamer moieties. In some embodiments, step a) is performed in the presence of divalent cations, such as Mg²⁺ at a concentration of at least 1 mM, for instance at a concentration of about 1 mM to 50 mM, for instance from 1 mM to 20 mM, such as a concentration of about 5 mM. In some other embodiments, step a) is performed in the absence of Mg²⁺; and more generally, in the absence of divalent cations.

Accordingly the binding buffer used in step a) may comprise NaCl at a concentration of about 100 mM to 200 mM and magnesium salt such as magnesium chloride (MgCl₂) at a concentration of about 1 mM to 50 mM and may have a pH of 5.0 or 6.9. Such a buffer may be suitable when the aptamer moieties present on the solid support are selected among the first subgroup of the invention as defined above.

An appropriate binding buffer for implementing step a), in particular when the aptamer moiety belongs to the first subgroup as defined above, may be a buffer comprising 50 mM of MOPS, 5 mM of MgCl₂ and 150 mM of NaCl, at pH 6.3.

When the aptamer moieties present on the affinity support are selected among the second subgroup of aptamers as defined above, the binding buffer may be devoid of Mg²⁺, and more generally of divalent cations. An appropriate binding buffer for implementing step a) may be thus a buffer comprising 50 mM of MOPS, and 150 mM of NaCl, at pH 6.3.

At the end of step a), and prior to step b), the affinity support may be washed with an appropriate washing buffer so as to remove the substances which are not specifically bound, but adsorbed onto the support. It goes without saying that the washing buffer does not significantly impair the complex between fibrinogen and the aptamer moiety while promoting desorption of the substances which do not specifically bind to the affinity support.

Thus, in some embodiments, the method of the invention comprises a step of washing the affinity support at the end of step a) and before step b). Any conventional washing buffer, well known to those skilled in the art, may be used. In some embodiments, the washing buffer as the same composition as that of the binding buffer used in step a). In other embodiments, the washing buffer may comprise the same components, but at different concentrations, as compared to the binding buffer used in step a). In some additional or alternative embodiments, the pH of the washing buffer is the same as that of the binding buffer.

The washing buffer may have a pH of less than 7, for instance from pH 5.0 to 6.9, preferably from 6.1 to 6.5, such as pH 6.3. The washing buffer may further comprise NaCl. Typically, the ionic strength of the washing buffer may be higher than that of the binding buffer. Indeed, the Applicants showed that, for certain aptamers of the invention, high ionic strength may not significantly impair the binding of fibrinogen to the aptamer moieties. In other words, the complex between fibrinogen and certain aptamers of the invention may be stable, even in the presence of high ionic strength. Thus, in some embodiments, the washing solution has a ionic strength higher than that of the binding buffer used in step a). In alternate or additional embodiments, the washing buffer may comprise a concentration of NaCl of at least 100 mM and up to 10 M. For instance the concentration of NaCl may be of about 100 mM to 5 M, preferably from 150 mM to 2 M. Optionally, the washing buffer further comprises divalent cations, in particular Mg²⁺, at a concentration of about 0.1 mM to 20 mM, preferably from 1 mM to 10 mM such as a concentration of about 5 mM. In some embodiments, the washing buffer is devoid of Mg²⁺ and more generally of divalent cations.

In some other or additional embodiments, the washing buffer may comprise at least one additional component, preferably selected among alkyl diols, in particular among ethylene glycol or propylene glycol. Indeed, for certain aptamers of the invention, the presence of alkyl diols such as ethylene glycol in the washing solution do not impair the complex between fibrinogen and the aptamer. The washing buffer may thus comprise an alkyl diol such as ethylene glycol or propylene glycol in an amount from 1% to 70% in weight, preferably from 10% to 60% in weight, such as 50% in weight.

For illustration only, the washing buffer comprises MOPS at 50 mM, NaCl at 2M, at pH 6.3 and optionally 50% of glycol in weight. Such a washing buffer may be appropriate to carry out the washing of an affinity support having thereon aptamer moieties belonging to the second subgroup of aptamers as described above. Another example of washing buffer is a solution comprising 50 mM of MOPS, 0.5 M NaCl and 5 mM of MgCl₂ at pH 6.3.

Step b) aims at releasing fibrinogen from the complex formed in step a). This release may be obtained by destabilizing the complex between fibrinogen and the aptamer moieties, i.e. by using conditions which decrease the affinity of the aptamers to fibrinogen. Noteworthy, the complex between the aptamer moiety and fibrinogen may be destabilized in mild conditions which are not susceptible to alter fibrinogen.

As explained above, the ability of the aptamers of the invention to bind to fibrinogen may depend on the pH of the medium. Increasing the pH above 7.0 may enable to promote the release of fibrinogen. Thus in certain embodiments, step b) is performed by increasing the pH above 7.0. Preferably, the pH of step b) is from 7.0 to 8.0, for instance from 7.2 to 7.8 such as a pH of 7.4. In other words, an elution buffer at pH above 7.0 may be used to promote the release of fibrinogen. For illustration only, an appropriate elution buffer may be a buffered solution of 50 mM MOPS at pH 7.4 and comprising 150 mM of NaCl.

As explained above, the aptamer capability of binding to fibrinogen may also vary depending on the presence of divalent cations, such as Mg²⁺. For instance, the binding of the aptamer moiety to fibrinogen may be promoted by the presence of Mg²⁺ Thus, the release of fibrinogen from the complex in step b) may be promoted by using an elution buffer devoid of divalent cations and/or comprising a divalent cation-chelating agent, such as EDTA or EGTA. For instance, the divalent cation-chelating agent may be present at a concentration of at least 1 mM and of at most 500 mM in the elution buffer used in step b). The use of a divalent cation-chelating agent may be appropriate for affinity support having thereon aptamers belonging to the first subgroup as described above.

In other embodiments, the binding of the aptamer moiety to fibrinogen may decrease in the presence of divalent cations such as Mg²⁺. Thus, in this embodiment, the elution buffer may comprise divalent cations, in particular Mg²⁺, at a concentration of about 0.1 mM to 20 mM, preferably from 1 mM to 10 mM such as a concentration of about 5 mM. Such elution may be suitable to release fibrinogen from complex formed with an aptamer moiety belonging to the second subgroup as defined above.

Another example of elution buffer which may be used in step b) is a solution of 50 mM MOPS at pH 7.4 comprising MgCl₂ at 2 M.

At the end of step c), the purified fibrinogen is typically obtained in the form of a liquid purified composition. This liquid purified composition may undergo one or several addition steps. Said liquid composition may be concentrated, and/or subjected to virus inactivation or removal, for instance by sterile filtration or by a detergent, diafiltration, formulation step with one or several pharmaceutically acceptable excipients, lyophilization, packaging, preferably under sterile conditions, and combinations thereof.

In a more general aspect, the method for purifying fibrinogen or the method for preparing a purified fibrinogen composition may comprise one or several additional steps including, without being limited to, chromatography step(s) such as exclusion chromatography, ion-exchange chromatography, multimodal chromatography, reversed-phase chromatography, hydroxyapatite chromatography, or affinity chromatography, precipitation step, one or several steps of filtration such as depth filtration, ultrafiltration, tangential ultrafiltration, nanofiltration, and reverse osmosis, clarification step, viral inactivation or removal step, sterilization, formulation, freeze-drying, packaging and combinations thereof.

In some embodiments, the method for purifying fibrinogen or the method for preparing a purified fibrinogen composition according to the invention is devoid any step of lyophilization or freeze-drying, desiccation, dehydration or drying step. In other words, the purified liquid fibrinogen composition obtained in step (c) may not be subjected to a treatment such as lyophilization (or freeze-drying), desiccation, dehydration or drying. The methods of the invention may comprise one or several (2, 3 or 4) of the following steps, which are performed after step (c):
a step of filtration such as ultrafiltration, tangential ultrafiltration or diafiltration, or osmosis,
a step of formulation by adding one or several pharmaceutical excipients to the composition,
a step of sterile filtration, for instance nanofiltration,
a step of packaging, preferably under sterile conditions.

In such embodiments, the resulting composition is liquid. It goes without saying that the invention also relates to the composition of fibrinogen obtained or obtainable by the methods of the invention as described herein.

In some additional embodiments, the method for purifying fibrinogen or the method for preparing a purified composition of fibrinogen comprises one of the following combinations of features:
combination 1: (i) the aptamer moiety is selected among aptamers of the first subgroup as defined above, (ii) step (a) is performed at pH 5.8 to 6.5, preferably 6.3, in the presence of Mg²⁺, and (iii) step (b) is performed at pH 7.0 to 8.0, preferably 7.4, optionally in the present of a divalent cation chelating agent and
combination 2: (i) the aptamer moiety is selected among aptamers of the second subgroup as defined above, (ii) step (a) is performed at pH 5.8 to 6.5, preferably 6.3, in the absence of Mg²⁺ and (iii) step (b) is performed at pH 7.0 to 8.0, preferably 7.4, in the presence of Mg²⁺.

In an additional aspect, the aptamers and the affinity ligands of the invention may be used in a blood plasma fractionation process. The blood plasma fractionation process may comprise several consecutive affinity chromatography steps, each affinity chromatography step enabling to recover a plasma protein of interest such as fibrinogen, immunoglobulin, albumin and other coagulation factors, such as vitamin K-dependent coagulation factors. The affinity ligands used in each step may be of any type, in particular aptamers. To that respect, the Applicant surprisingly showed that plasma proteins such as fibrinogen, albumin, and immunoglobulin, can be recovered and purified from blood plasma by performing successive aptamer-based affinity chromatography steps. Noteworthy, blood plasma fractionation process comprising successive aptamer-based affinity chromatography steps enable to obtain fibrinogen concentrate and immunoglobulin concentrate with a protein purity of at least 96%, and even of at least 99% and with yields of about 9-12 g per plasma litre for immunoglobulins and 2-4 g per plasma litre for fibrinogen. The Applicant further showed that these good yields and purity rates can be achieved from raw blood plasma. In other words, the aptamer-based affinity chromatography steps can be performed on raw blood plasma without any pre-treatment such as ethanol fractionation (Cohn process), cryoprecipitation, caprylate fractionation or PEG precipitation. Notably, such fractionation process enables to avoid temporary intermediary cold storages.

A further object of the invention is thus a blood plasma fractionation process comprising:

(a) an affinity chromatography step to recover fibrinogen wherein the affinity ligand is an aptamer which specifically bind to fibrinogen, and
(b) an affinity chromatography step to recover immunoglobulins (Ig) wherein the affinity ligand is preferably an aptamer which specifically bind to immunoglobulins, wherein the affinity chromatography steps (a) and (b) can be performed in any order.

Preferably, immunoglobulins of G isotype are recovered in step (b).

The affinity chromatography step for recovering fibrinogen can be performed before the affinity chromatography to recover Ig and vice versa. Accordingly, in some embodiments, the blood plasma fractionation process comprises the steps of:
subjecting blood plasma or a derivative thereof to an affinity chromatography step, wherein the affinity ligand is an aptamer which specifically binds to fibrinogen, and
subjecting the non-retained fraction, which is substantially free from fibrinogen, to an affinity chromatography step, wherein the affinity ligand is an aptamer which specifically binds to immunoglobulin.

It goes without saying that the above steps may comprise recovering fibrinogen and Ig retained on the affinity support, respectively.

In some other embodiments, the blood plasma fractionation process comprises the steps of:
subjecting blood plasma or a derivative thereof to an affinity chromatography step, wherein the affinity ligand is an aptamer which specifically binds to Ig, and
subjecting the non-retained fraction, which is substantially free from Ig, to an affinity chromatography step, wherein the affinity ligand is an aptamer which specifically binds to fibrinogen.

It goes without saying that the above steps may comprise recovering Ig and fibrinogen retained on the affinity support, respectively.

In the process of the invention, the starting composition can be a blood plasma or derivatives thereof. Derivatives of blood plasma encompass, without being limited to, a clarified blood plasma, a lipid-depleted blood plasma, a blood plasma cryoprecipitate, a supernatant of a blood plasma cryoprecipitate, a plasma fraction and the like. In some embodiments, the starting composition is a raw blood plasma.

Preferably, immunoglobulins of the G isotype are recovered. Immunoglobulins of G isotype encompass IgG1, IgG2, IgG3 and IgG4. In some embodiments, the aptamer directed against the immunoglobulin is able to specifically bind to IgG, regardless IgG subclasses. In some embodiments, several types of anti-IgG aptamers are used so as to recover all the subclasses of IgG. Preferably, the IgG fraction recovered in the fractionation process of the invention has a subclasses distribution close to that of the starting blood plasma, namely comprises from 50% to 70% of IgG1, from 25% to 35% of IgG2, from 2% to 8% of IgG3 and 1 to 8% of IgG4.

In some embodiments, the blood plasma fractionation process of the invention comprises one or several additional steps, in particular (c) a step of purifying albumin.

Purified albumin can be recovered by any conventional methods such as chromatography including affinity chromatography, ion-exchange chromatography, and ethanol precipitation followed by filtration.

For instance, step (c) can be an affinity chromatography step wherein the affinity ligand is an aptamer which specifically bind to albumin.

When step (c) is present, steps (a), (b) and (c) can be performed in any order. In some embodiments, step (c) is performed on the non-retained fraction obtained from step (a) or step (b).

Any type of chromatography technology can be used to implement steps (a), (b) and (c) in the process of the invention, such as batch chromatography, Simulated Moving Bed (SMB) Chromatography or Sequential Multicolumn Chromatography (SMCC). Preferred chromatography technologies are those comprising the use of multi-columns such as SMB chromatography and SMCC. Multi-column chromatography technology is based on the use of several small columns, comprising the same stationary phase, instead of one single chromatography column as in the case of batch chromatography. These small columns are typically connected in series.

Examples of multicolumn chromatography process are described for instance in WO2007/144476, WO2009/122281 and WO2015136217, the disclosure of which being incorporated herein by reference.

In some embodiments, the blood plasma fractionation process of the invention comprises at least one multicolumn chromatography step, said step being preferably step (a).

In some other embodiments of the fractionation process of the invention, steps (a) and (b) are multicolumn chromatography steps, in particular SMCC. In some additional or alternate embodiments, step (c) is present and is a multicolumn chromatography step. In some additional steps, all the chromatography steps of the blood plasma process of the invention are multicolumn chromatography steps, in particular SMCC.

In some alternate or additional embodiments, the chromatography column(s) used in steps (a) and/or (b) and/or (c) is/are radial chromatography column(s). Appropriate radial columns encompass, without being limited to, radial columns having a ratio of the largest external diameter surface to the smallest inner diameter surface of 2.

In some embodiments, the binding buffers used in steps (a), (b) and in the optional step (c) are such that the chromatography steps can be performed consecutively, without any pre-treatment steps such as a dialysis or diafiltration step between them. For instance, when step (a) is performed before step (b), the non-retained fraction obtained from step (a) can be used in step (b) without any pre-treatment such as diafiltration.

In some embodiments, the same binding buffer conditions are used in step (a), step (b) and optional step (c). In some other embodiments, the buffers used in steps (a), (b) and in the optional step (c) are such that minor intermediary steps are performed before carrying out the subsequent chromatography step. Minor intermediary steps encompass pH adjustment, conductivity adjustment, and/or ionic strength adjustment of the non-retained fraction resulting from the precedent chromatography step as well as the addition and/or the removal of a specific excipient in said non-retained fraction.

The blood plasma fractionation process can comprise one or several additional steps including, without being limited to, chromatography step to remove anti-A and/or anti-B antibodies, ultrafiltration, tangential ultrafiltration, nanofiltration, reverse osmosis, clarification, viral inactivation step, virus removal step, sterilization, polishing steps such as formulation, or freeze-drying and combinations thereof. The process of the invention may also comprise one or several additional steps aiming at preventing and/or removing the fouling of the chromatography columns such as sanitization with an alkaline solution, e.g. with sodium hydroxide solution.

The invention also relates to a purified composition of fibrinogen obtainable or obtained by a method for preparing a purified fibrinogen composition according to the invention or by the blood plasma fractionation process according to the invention.

A further object of the invention is a purified composition of fibrinogen which comprises at least 90% by weight, preferably at least 91%, 92%, 93%, 94, 95%, 96%, 97% 98%, 99%, 99.1%, 99.2%, 99.3%, 99.4%, 99.5%, 99.6%, 99.7%, 99.8%, or 99.9% by weight as compared to the total weight of proteins present in said composition. In some embodiments, the purified composition of fibrinogen comprises human plasmatic fibrinogen, e.g. fibrinogen obtained from human plasma or human plasma fraction. In such an embodiment, said composition comprises at most 10%, preferably at most 9%, 8%, 7%, 6%, 5%, 4% 3%, 2%, 1%, 0.9%, 0.8%, 0.7%, 0.6%, 0.5%, 0.4%, 0.3%, 0.2% or 0.1% by weight of other plasma proteins, in particular of other human coagulation factors. In some embodiments, the composition is substantially devoid of human coagulation factors other than human fibrinogen. In some additional or alternate embodiments, said composition is devoid of factor XIII.

In some embodiments, the purified composition of fibrinogen is obtained from human plasma or derivatives thereof such as human plasma fractions or prepurified fibrinogen composition, and comprises:
less than 10 μg, preferably less than 5 μg of fibronectin per mg of fibrinogen, and/or
less than 0.01 mUI, preferably less than 5 μUI of factor II per mg of fibrinogen, and/or
less than 0.6 mUI, preferably less than 0.4 mUI of factor XI per mg of fibrinogen, and/or
less than 5 mUI, preferably less than 3 mUI of factor XIII per mg of fibrinogen, and/or
less than 0.1 μg, preferably less than 0.05 μg, and even less than 15 ng of plasminogen per mg of fibrinogen.

The purified composition fibrinogen is preferably liquid and stable. The feature “stable” is defined further below.

In some embodiments, the purified composition of fibrinogen comprises human recombinant fibrinogen, e.g. human fibrinogen produced in a recombinant host such as recombinant cell or a transgenic animal. In such an embodiment, said composition comprises at most 10%, preferably at most 9%, 8%, 7%, 6%, 5%, 4% 3%, 2%, 1%, 0.9%, 0.8%, 0.7%, 0.6%, 0.5%, 0.4%, 0.3%, 0.2% or 0.1% by weight of other proteins, in particular of non-human proteins from the recombinant host. In some embodiments, the composition is substantially devoid of non-human proteins. In some additional or alternate embodiments, said composition is devoid of any non-human homolog of fibrinogen which may be found in the recombinant host.

The invention also relates to a pharmaceutical composition comprising a purified composition of human fibrinogen such as recombinant human fibrinogen or human plasmatic fibrinogen as defined above, in combination with one or more pharmaceutically acceptable excipients. Said pharmaceutical composition as well as the liquid purified composition of fibrinogen according to the invention can be used in the treatment of coagulation disorders, in particular in the treatments of congenital or acquired deficiency in fibrinogen (hypo-, dys- or afibrinogenaemia). The composition of the invention may be used in the management of post-traumatic or post-surgical acute bleedings or in the management of fibrinogen deficiency resulting from acute renal failure.

Composition Comprising Fibrinogen Stable in Liquid Form According to the Invention

Surprisingly, the Applicant showed that compositions comprising fibrinogen obtained by the methods described herein, in particular by the method for purifying fibrinogen from a starting composition described above, were particularly stable under storage, when formulated in liquid form with a minimal amount of excipients (see Example 6 below).

Thus, in an additional aspect, the Invention also relates to a composition comprising fibrinogen which is stable in liquid form.

The term “stable” refers to the physical and/or chemical stability of the composition comprising fibrinogen. The term “physical stability” refers to the reduction or the absence of formation of insoluble or soluble aggregates of the dimeric, oligomeric or polymeric forms of fibrinogen, to the reduction or the absence of formation of precipitate, and to the reduction or the absence of any structural denaturation of the molecule.

The term “chemical stability” refers to the reduction or the absence of any chemical modification of the composition comprising fibrinogen during storage, in the liquid state, under accelerated conditions.

A stability test can be carried out in various temperature, humidity and light conditions. Preferably, in the context of the present invention, the stability test can last at least 1 week, preferably at least 1 month, e.g. at least 2 months, at least 3 months, at least 4 months, at least 5 months, at least 6 months.

Typically, the stability parameters, as defined below, are measured:
before the stability testing of a composition comprising fibrinogen; so as to determine the initial level of the parameters; and
during or at the end of said stability test,
   given that said stability test can last at least 1 week, preferably at least 1 month, e.g. at least 2 months, at least 3 months, at least 4 months, at least 5 months, or at least 6 months.

In certain embodiment, the stability of the composition comprising fibrinogen is evaluated by measuring the coagulation activity of the fibrinogen relative to its antigenic activity (also called specific activity). In a preferred embodiment, the stable fibrinogen composition has a fibrinogen clotting activity/fibrinogen antigenic activity ratio more than 0.5, preferably more than 0.6, more than 0.7, more than 0.8, more than 0.9, even more preferably roughly equal to 1.0. In some embodiments, the fibrinogen clotting activity/fibrinogen antigenic activity ratio after the stability test is at least 60%, preferably at least 70%, 80%, 90%, 95%, 98% or 99% of the initial fibrinogen clotting activity/fibrinogen antigenic activity ratio in the composition before the stability test.

By “fibrinogen clotting activity” is meant the measurement of functional fibrinogen by a coagulation technique, determined according to the von Clauss method. Clotting activity is expressed in g/L of fibrinogen solution. This technique is known to the one skilled in the art, who may refer to the publication Von Clauss, A. (1957) Gerinnungsphysiologische schnellmethode zur bestimmung des fibrinogens. Acta Haematologica, 17, 237-246.

By “antigenic fibrinogen” is meant the amount of fibrinogen, whether active or inactive, measured by a nephelometric method. The amount of antigenic fibrinogen is expressed in g/L. In other or alternative embodiments, the stability of the composition comprising fibrinogen may be also evaluated by SDS-PAGE measurement of retention of the alpha, beta and gamma chains of fibrinogen, preferably before and after a stability test as defined in the context of the present invention. Thus, a fibrinogen composition may be advantageously considered stable if:
all of the alpha chains are at least 50% retained, preferably at least 60% retained, preferably at least 70% retained, preferably at least 80% retained, preferably at least 90% retained; more preferably roughly 100% retained, and/or
all of the beta chains are at least 50% retained, preferably at least 60% retained, preferably at least 70% retained, preferably at least 80% retained, preferably at least 90% retained; more preferably roughly 100% retained, and/or
all of the gamma chains are at least 50% retained, preferably at least 60% retained, preferably at least 70% retained, preferably at least 80% retained, preferably at least 90% retained; more preferably roughly 100% retained.

The percentage of alpha chain retention may be calculated from the amount of alpha chains detected in the sample during or at the end of the stability test as compared to the initial amount of alpha chains in the sample before the stability test. This is the same for the gamma chain and the beta chain retention percentages. The amount of a given type of chain may be assessed for instance from the relative intensity of the band(s) in the SDS-PAGE electrophoresis gel, in reduced conditions, said band(s) corresponding to the molecular weight(s) of said given type of chain.

In some other or additional embodiments, the stability of the composition comprising fibrinogen may be also evaluated by SDS-PAGE measurement of Aα1 chain in the sample before the stability test and during or at the end of the stability test. The fibrinogen composition may be considered as stable if the amount of Aα1 chain after the stability test is at least 50%, preferably at least 60%, 70%, 80% and even at least 90% of the amount of Aα1 chain in the composition before the stability test. The amount of Aα1 chain may be assessed for instance from the relative intensity of the band in the SDS-PAGE electrophoresis gel, in reduced conditions, which corresponds to the molecular weight of Aα1 chain.

In a particular embodiment, a fibrinogen composition is considered as stable in liquid form if said composition shows at least one (for instance 1, 2, or 3) of the following features:
The amount of Aα1 chain, determined by SDS-PAGE in reduced conditions, after the stability test is at least 50%, preferably at least 60%, 70%, 80% and even at least 90% of the amount of Aα1 chain in the composition before the stability test,
At least 70%, preferably at least 80% or 90% of all of the alpha chains, determined by SDS-PAGE in reduced conditions, are retained after the stability test.
The initial fibrinogen clotting activity/fibrinogen antigenic activity ratio is more than 0.7, preferably more than 0.8, or 0.9, and even about 1 and the fibrinogen clotting activity/fibrinogen antigenic activity ratio after the stability test is at least 60%, preferably at least 70%, 80%, 90%, 95%, 98% or 99% of the initial fibrinogen clotting activity/fibrinogen antigenic activity ratio in the composition before the stability test.

In said embodiment, the stability test may be performed by keeping the fibrinogen composition at a temperature of 5° C., during at least one month.

Other possible parameters to assess the stability of the liquid composition may encompass the variation of pH and/or the variation of the osmolality before and after the stability test. In some embodiments, the pH of the liquid composition at the end of the stability test is included in a the range [pH₀-1, pH₀+1], preferably [pH₀-0.5, pH₀+0.5], pH₀ being the initial pH of the composition prior to the stability test. In some other or additional embodiments, the osmolality after the stability test is from 70% to 130%, preferably from 80% to 120%, and even from 90% to 110% of the initial osmolality of the composition prior to the stability test.

In an advantageous embodiment of the invention, the composition comprising fibrinogen is stable for at least 1 month, at least 2 months, at least 3 months, at least 4 months, at least 5 months, at least 6 months at 4° C.

According to the present aspect of the invention, by “composition according to the invention” may be meant the composition comprising fibrinogen, said composition being stable in liquid form. Preferably, said composition consists of fibrinogen, arginine and citrate.

In a particular embodiment, “fibrinogen composition in liquid form” may refer to a composition comprising fibrinogen in solution, preferably which has not been subjected to a lyophilization, desiccation, dehydration or drying step, and thus which does not need to be reconstituted before use. In some embodiments, said fibrinogen composition may be a ready-to-use composition, namely it can be directly injected to the patient.

Another object of the invention is thus a pharmaceutical composition comprising fibrinogen which is stable in liquid form, said pharmaceutical composition being preferably a non-reconstituted composition, e.g. a non-lyophilized and a non-reconstituted composition.

Another object of the invention is a pharmaceutical composition comprising fibrinogen and one or more pharmaceutically acceptable excipients, which is stable in liquid form.

The term “pharmaceutically acceptable excipient” refers to any excipient advantageously usable for formulating human proteins, such as substances selected from salts, amino acids, sugars, surfactants or any other excipient.

The pharmaceutically acceptable excipients of the invention may exclude isoleucine, glycine and NaCl.

Advantageously, the pharmaceutically acceptable excipients according to the invention include arginine and/or citrate. The Applicant demonstrated that it was possible to obtain compositions which are particularly stable over time in liquid form comprising fibrinogen, arginine and citrate. Such a composition is optimal, because it limits the number of excipients and thus the risk of side effects due to the components of the formulation while allowing the ready-to-use composition to be stored in liquid form.

Thus, in a preferred embodiment, the invention relates to a composition comprising, preferably consisting of, fibrinogen, arginine and citrate, e.g. citrate salt such as trisodium citrate, and which is stable in liquid form. The liquid form of the composition is preferably an aqueous solution. In other words, the composition of the invention in liquid form comprises, or consists in, fibrinogen, arginine and citrate salt in water. The pH of the liquid form of the composition according to the invention is from 6.0 to 8.0, preferably from 6.5 to 7.5 such as about 7.0.

Preferably, said fibrinogen is human fibrinogen.

According to the invention, several sources of raw material containing fibrinogen can be used.

The fibrinogen composition can thus be derived from plasma, also called plasma fractions, from cell culture supernatant or from body fluids, e.g. milk, of transgenic animals.

In a preferred embodiment, the composition of the invention has not undergone any preliminary lyophilization, desiccation, dehydration or drying step.

In a preferred embodiment, the composition of the invention has not undergone any preliminary step of reconstitution of a lyophilizate.

In a particular embodiment, the composition according to the invention is a plasma fraction e.g. a human plasma fraction, preferably a plasma fraction obtained from pre-purified plasma, preferably a human plasma fraction.

By “plasma fraction obtained from prepurified plasma” is meant any part or subpart of human plasma that has been subjected to one or more purification steps. Said plasma fractions thus include the supernatant of cryoprecipitated plasma, plasma cryoprecipitate (resuspended), fraction I obtained by ethanol fractionation (according to the Cohn or Kistler & Nitschmann method), chromatography eluates and unadsorbed chromatography column fractions, including multiple-column chromatography, and filtrates.

In some embodiments, the composition according to the invention is derived from a plasma fraction obtained from cryosupernatant or from resuspended cryoprecipitate.

According to the invention, “supernatant of cryoprecipitated plasma” or “cryosupernatant” refers to the liquid phase obtained after thawing frozen plasma (cryoprecipitation). Notably, the cryosupernatant can be obtained by freezing plasma at a temperature between −10° C. and −40° C., then gentle thawing at a temperature between 0° C. and +6° C., preferably between 0° C. and +1° C., followed by centrifugation of the thawed plasma to separate the cryoprecipitate and the cryosupernatant. The cryoprecipitate is a concentrate of fibrinogen, fibronectin, von Willebrand factor and factor VIII, while the cryosupernatant contains complement factors, vitamin K-dependent factors such as protein C, protein S, protein Z, factor II, factor VII, factor IX and factor X, fibrinogen, immunoglobulins and albumin.

In a particular embodiment, the composition of the invention derives from plasma not previously depleted of proteins such as immunoglobulins or albumin.

In a preferred embodiment, the composition according to the invention is derived from a chromatography eluate or from a non-adsorbed column chromatography fraction, including multiple-column chromatography. In an even more preferred embodiment of the invention, the composition according to the invention is derived from a chromatography eluate or from a non-adsorbed column chromatography fraction, excluding multiple-column chromatography.

The chromatography is preferably an affinity chromatography purification step carried out by using affinity ligands such as aptamers directed against fibrinogen.

In a particular embodiment, the composition of the invention derives from a chromatography eluate, said chromatography being an affinity chromatography wherein the affinity ligand is an anti-fibrinogen aptamer according to the invention as described above. For instance, said aptamer comprises a polynucleotide having at least 70% of sequence identity with the nucleotide sequence of SEQ ID No 66. Alternatively, said aptamer may comprise the nucleotide moiety of formula (III) as defined above.

As mentioned above, said affinity chromatography may be performed on any type of composition comprising fibrinogen, such as plasma and plasma fractions, included plasma fractions non-depleted in albumin or immunoglobulins.

In some embodiments, the composition of the invention is obtainable or is obtained by a process comprising the following steps:
an affinity chromatography purification step;
at least one biosafety step, such as virus inactivation or removal, for instance by sterile filtration or by a detergent; and
a formulation step into liquid form.

In certain embodiment, the composition of the invention is obtainable or is obtained by a process comprising the following steps:
providing a blood plasma or a cryosupernatant fraction of blood plasma,
purifying said blood plasma or said cryosupernatant fraction of blood plasma by separation on affinity chromatography gel preferably using affinity ligands selected from aptamers, preferably an aptamer directed against fibrinogen as described herein,
collecting the purified adsorbed fraction comprising fibrinogen, and
optionally, adding pharmaceutically acceptable excipients, preferably arginine and/or citrate such as citrate salt

In a particular embodiment of the invention, the stable liquid composition comprising fibrinogen is obtained or obtainable by a process comprising the following steps:
providing a cryosupernatant fraction of blood plasma,
precipitating the cryosupernatant with 8% ethanol to obtain a fibrinogen-enriched fraction,
resuspending the fibrinogen-enriched plasma fraction and then purifying said fraction by separation on affinity chromatography gel preferably using oligonucleotide ligands such as aptamers, for instance as described herein,
collecting the purified adsorbed fraction comprising fibrinogen, and
optionally, adding pharmaceutically acceptable excipients, preferably arginine and/or citrate such as citrate salt.

The affinity chromatography step is preferably performed with an anti-fibrinogen aptamer, including the anti-fibrinogen aptamers as described herein. Preferred conditions to implement the chromatography step are described herein, in particular in pages 30-34. As mentioned above, the process may comprise additional steps e.g. biosafety step such as sterile filtration and virus inactivation.

In a particular embodiment, the process further comprises a step of storage of the composition for at least 3 months at 4° C. In another or additional embodiment, the process may comprise a step of packaging of the composition, for instance in a vial, in a cartridge or a device for injection, such as a pre-mounted-syringe.

In some embodiments, the composition according to the invention is free of proteases and/or fibrinolysis activators.

By “fibrinogen composition free of proteases and/or fibrinolysis activators” is meant that the fibrinogen composition has undergone one or more steps so as to remove proteases, such as thrombin, prothrombin, plasmin and plasminogen, so that the residual amount of proteases and/or fibrinolysis activators is:
drastically reduced in comparison with the prepurified fibrinogen solution before the chromatography step, and/or
null, and/or
below the detection thresholds of the methods commonly used by persons skilled in the art.

Advantageously, the residual prothrombin level is less than 5 μIU/mg fibrinogen, and/or the plasminogen level is less than 50 ng/mg fibrinogen such as 15 ng/mg fibrinogen.

In a particular embodiment of the invention, the composition according to the invention is thus free of proteases such as thrombin and/or plasmin or the corresponding proenzymes thereof prothrombin (coagulation factor II) and/or plasminogen, which are potentially activable zymogens.

In a particular embodiment of the invention, the fibrinogen composition according to the invention is free of protease inhibitors and/or antifibrinolytics.

By “protease inhibitors and/or antifibrinolytics” is meant any molecule having antiprotease activity, notably any molecule having serine protease inhibitor and/or antifibrinolytic activity, in particular any molecule having thrombin inhibitor and/or antiplasmin activity, in particular hirudin, benzamidine, aprotinin, phenylmethylsulfonyl fluoride (PMSF), pepstatin, leupeptin, antithrombin III optionally associated with heparin, alpha 2-macroglobulin, alpha 1-antitrypsin, hexanoic or epsilon aminocaproic acid, tranexamic acid, and/or alpha 2-antiplasmin.

In a particular embodiment of the invention, the fibrinogen composition according to the invention is free of hirudin and/or benzamidine and/or aprotinin and/or PMSF and/or pepstatin and/or leupeptin and/or antithrombin III optionally associated with heparin and/or alpha 2-macroglobulin and/or alpha 1-antitrypsin and/or hexanoic and/or epsilon aminocaproic acid and/or tranexamic acid and/or alpha 2-antiplasmin.

In a particular embodiment of the invention, the fibrinogen composition according to the invention is free of metal ions. In a particular embodiment of the invention, the composition according to the invention is advantageously free of calcium. In a particular embodiment of the invention, the fibrinogen composition according to the invention is free of isoleucine, glycine and/or NaCl.

In a particular embodiment of the invention, the fibrinogen composition according to the invention is free of albumin.

Advantageously, the composition according to the invention has a purity greater than or equal to 70%, preferably greater than or equal to 75%, 80%, 85%, 90%, 95%, 96%, 97%, 98%, 99%.

In a particular embodiment of the invention, the composition according to the invention comprises no other copurified proteins, advantageously not FXIII and/or fibronectin. In another particular embodiment of the invention, the fibrinogen composition according to the invention may also comprise one or more accompanying proteins, optionally copurified. In a particular embodiment of the invention, the composition according to the invention advantageously comprises FXIII.

The composition according to the invention is subjected, directly after purification, to the steps of pharmaceutical formulation in liquid form: formulation, sterile filtration and distribution into a container (flask or other storage/administration device).

Particularly advantageously, the composition according to the invention is not subjected to a lyophilization, desiccation, dehydration or drying step. In other words, the composition of the invention may be obtained by a process devoid of any lyophilization, desiccation, dehydration or drying step.

Particularly advantageously, the composition according to the invention is thus in liquid form without having undergone a step of reconstitution of a lyophilizate.

Particularly advantageously, the composition according to the invention is in liquid form, and thus comprises water in addition to possible pharmaceutically acceptable excipients.

In a particular embodiment of the invention, the composition in liquid form comprises citrate and/or arginine, preferably less than 300 mM arginine. The citrate is typically a citrate salt such as trisodium salt which may be present at a concentration from 1 to 100 mM, preferably from 1 to 50 mM such as around 5 to 15 mM in the composition.

Advantageously, the composition according to the invention is particularly suitable for intravenous administration. It goes without saying that the composition of the invention can be used in therapy. For instance, the composition of the invention may be used in
the treatment of congenital or acquired deficiencies in fibrinogen (hypo-, dys- or afibrinogenaemia).
the management of post-traumatic or post-surgical acute hemorrhages or
in the management of fibrinogen deficiency resulting from acute renal failure.

Method for Obtaining the Aptamers of the Invention

The Applicants carried-out several SELEX strategies described in the prior art to identify aptamers against human fibrinogen. None of these strategies succeeded and standard SELEX led to the identification of aptamers directed against a contaminant accounting for less than 1% in the purified fibrinogen composition used for implementing it.

In that context, the Applicant performed extensive researches to develop a new method for obtaining aptamers directed against “SELEX-resistant” proteins such as fibrinogen.

The Applicant conceived a new SELEX process which enables to obtain aptamers displaying high binding affinity for “SELEX-resistant” proteins, and which may be used as affinity ligands in purification process. This new SELEX process is characterized by a selection step which is performed in conditions of pH suitable to create “positive patches” on the surface of the protein target. In other words, the process conceived by the Applicant is based on the enhancement of the local interactions between the potential aptamers and the targeted protein by promoting positive charges on a surface domain of the protein. This method can be implemented for proteins having one or several surface histidines, such as fibrinogen. The pH of the selection step (.i.e. the step wherein the protein target is contacted with the candidate mixture of nucleic acids) should be selected so as to promote the protonation of at least one surface histidine of the protein target. In the case of fibrinogen, the applicant showed that an appropriate pH for the selection step is a slightly acid pH.

Accordingly, the invention also relates to a method for obtaining an aptamer which specifically binds to fibrinogen, said method comprising:
a) contacting fibrinogen with a candidate mixture of nucleic acids at a pH lower than 7.0, preferably from 5.8 to 6.8,
b) recovering nucleic acids which bind to fibrinogen, while removing unbound nucleic acids,
c) amplifying the nucleic acids obtained in step (b) to yield to a candidate mixture of nucleic acids with increased affinity to fibrinogen, and
d) repeated steps (a), (b), (c) until obtaining one or several aptamers against fibrinogen.

In step (a), the candidate mixture of nucleic acids is generally a mixture of chemically synthesized random nucleic acid. The candidate mixture may comprise from 10⁸ to 10¹⁸, typically about 10¹⁵ nucleic acids. The candidate mixture may be a mixture of DNA nucleic acids or a mixture of RNA nucleic acids. In some embodiments, the candidate mixture consists of a multitude of single-stranded DNAs (ssDNA), wherein each ssDNA comprises a central random sequence of about 20 to 100 nucleotides flanked by specific sequences of about 15 to 40 nucleotides which function as primers for PCR amplification. In some other embodiments, the candidate mixture consists of a multitude of RNA nucleic acids, wherein each RNA comprises a central random sequence of about 20 to 100 nucleotides flanked by primer sequences of about 15 to 40 nucleotides for RT-PCR amplification. In some embodiments, the candidate mixture of nucleic acids consists of unmodified nucleic acids, this means that the nucleic acids comprise naturally-occurring nucleotides only. In some other embodiments, the candidate mixture may comprise chemically-modified nucleic acids. In other words, the nucleic acids may comprise one or several chemically-modified nucleotides. In preferred embodiments, the candidate mixture consists of single-stranded DNAs.

Step a) is performed in conditions favourable for the binding of fibrinogen with nucleic acids having affinity for said fibrinogen. Preferably, the pH of step a) is from 6.0 to 6.6, such as 6.1, 6.2, 6.3, 6.4 and 6.5. An appropriate pH for step a) is for instance, 6.3±0.1. Such pH enables to protonate at least one surface histidine of fibrinogen. Step (a) may be performed in a buffered aqueous solution. The buffering agent may be selected from any buffer agents enabling to obtain the desired pH and compatible with the protein targets and the nucleic acids mixture. The buffer agent may be selected from, without being limited to, 3-(N-morpholino)propanesulfonic acid (MOPS), 2-(N-morpholino)ethanesulfonic acid (MES), HEPES, Bis-TRIS, citrate and acetate. The buffering agent may be present at a concentration of about 5 mM to 1 M, for instance from 10 mM to 500 mM, for instance from 10 mM to 200 mM such as about 50 mM.

In some embodiments, fibrinogen may be present in free-state in step (a). In some other embodiments, fibrinogen may be immobilized on a solid support in order to make easier the subsequent separation of the complex formed by the protein target with certain nucleic acids and the unbound nucleic acids in step (b). For instance, fibrinogen may be immobilized onto magnetic beads, on solid support for chromatography such as sepharose or agarose, on microplate wells and the like. Alternatively, fibrinogen may be tagged with molecules useful for capturing of the complex in step (b). For instance, fibrinogen may be biotinylated.

Step (b) aims at recovering nucleic acids which bind to fibrinogen in step (a), while removing unbound nucleic acids. Typically, step (b) comprises separating the complex formed in step (a) from unbound nucleotides, and then releasing the nucleic acids from the complex whereby a new mixture of nucleic acids with increased affinity to the target protein is obtained.

The separation of the complex from the unbound nucleic acids may be performed by various methods and may depend on the features of fibrinogen. These methods include without being limited to, affinity chromatography, capillary electrophoresis, flow cytometry, electrophoretic mobility shift, Surface Plasmon resonance (SPR), centrifugation, ultrafiltration and the like. The skilled artisan may refer to any separation methods described in the state in the art for SELEX processes, and for instance described in Stoltenburg et al. Biomolecular Engineering, 2007, 24, 381-403, the disclosure of which being incorporating herein by reference. As illustration only, if fibrinogen is immobilized on a support, the separation may be performed by recovering the support, washing the support with an appropriate solution and then releasing nucleic acids from the complex immobilized on the support. If fibrinogen has been incubated in free-state with the candidate mixture, the separation of the nucleic acid-protein complex from unbound nucleic acids can be performed by chromatography by using a stationary support able to specifically bind to fibrinogen or the possible tag introduced on fibrinogen, whereby the complexes are retained on the support and the unbound nucleic acids flow out. For instance, one may use a stationary phase having thereon antibodies directed against the target protein. Alternatively, the partitioning may be performed by ultrafiltration on nitrocellulose filters with appropriate molecular weight cut-offs. Once the complexes separated from unbound nucleic acids, the nucleic acids which bind to fibrinogen are released from the complexes. The release can be performed by denaturing treatments such as heat treatment or by elution. Preferably, said nucleic acids are recovered by using an elution buffer able to dissociate the complex. The dissociation may occur by increasing the ionic strength or by modulating the pH in the elution buffer as compared to the buffered solution used in step a). For instance, if the pH of step (a) is 6.4, the pH of the elution buffer may be from 6.9 to 7.9, such as 7.4.

In a particular embodiment, step b) comprises the steps of separating the complex formed in step (a) from unbound nucleic acids, and then releasing the bound nucleic acids from the complex. The dissociation of the complex between fibrinogen and bound nucleic acids can be performed by increasing the pH above 7.0 in step b). Typically, in step b) the nucleic acids are recovered by dissociating the complex between fibrinogen and the nucleic acids at a pH above 7.0, for instance from pH 7.0 to 8.0, preferably from pH 7.2 to 7.8, more preferably from 7.2 to 7.6, such as 7.4. Preferably, in step b), the complex is immobilized on a solid support by the mean of fibrinogen. This means that fibrinogen is immobilized by covalent or non-covalent interactions on the solid support as described above. After an optional washing step, typically with the buffer used in step a), the complex between the nucleic acids and fibrinogen can be dissociated with an elution buffer having a pH from pH 7.0 to 8.0, preferably from pH 7.2 to 7.8, more preferably from 7.2 to 7.6, such as 7.4. The nucleic acids of interest are thus recovered in the elution buffer.

In alternate or additional embodiments, the elution buffer may comprise EDTA or detergent such as SDS, or urea. For instance, the elution buffer may comprise EDTA at a concentration of about 100 mM to 500 mM.

In step (c), the nucleic acids recovered in step (b) are amplified so as to generate a new mixture of nucleic acids. This new mixture is characterized by an increased affinity to the target protein as compared to the starting candidate mixture.

Step (a), (b) and (c) form together a round of selection. As indicated in step (d), this round of selection can be repeated several times, typically 6-20 times until obtaining an aptamer or a pool of aptamers directed against the target protein. It goes without saying that the step (a) of round “N” is performed with the mixture of nucleic acids obtained in step (c) of the round “N-1”. At the end of each selection round, the complexity of the mixture obtained in step (c) is reduced and the enrichment in nucleic acids which specifically bind to the target protein is increased.

The conditions for implementing step (a), (b) and (c) may be the same or may be different from one round of selection to another. In particular, the conditions of step (a) (e.g. the incubation conditions of the target protein with the mixture of nucleic acids) can change. For instance, step (a) of round “N” can be performed in more drastic conditions than in round “N+1” in order to direct the selection to aptamers having the highest affinity for fibrinogen. Typically, such result can be obtained by increasing the ionic strength of the buffer used in step (a).

The method of the invention may comprise one or several additional steps. The method of the invention may comprise counter-selection or substractive selection rounds. The counter-selection rounds may aim at eliminating nucleic acids which cross-react with other entities or directing the selection to aptamers binding to a specific domain of fibrinogen.

The method of the invention may comprise one or several of the following steps:
a step of cloning the aptamer pool,
a step of sequencing an aptamer,
a step of producing an aptamer, for instance by chemical synthesis,
a step of identifying consensus sequences in the pool of aptamers, for instance by sequence alignment,
a step of optimizing the sequence of an aptamer,

In some embodiments, the method of the invention may comprise the following additional steps:
sequencing an aptamer obtained in step (c)
optimizing said aptamer, and
producing the optimized aptamer, preferably by chemical synthesis.

The optimization of the aptamer may comprise the determination of the core sequence of the aptamer, i.e. the determination of the minimal nucleotide moiety able to specifically bind to fibrinogen. Typically, truncated versions of the aptamer are prepared so as to determine the regions which are not important in the direct interaction with fibrinogen.

The binding capacity of the starting aptamer and the truncated versions may be assessed by any appropriate methods such as SPR.

Alternatively or additionally, the sequence of the aptamer may be subjected to mutagenesis in order to obtain aptamer mutants, for instance with improved affinity or specificity as compared to their parent aptamer. Typically one or several nucleotide modifications are introduced in the sequence of the aptamer. The resulting mutants are then tested for their ability to specifically bind to fibrinogen, for example by SPR or ELISA-type assay.

In additional or alternate embodiments, the optimization may comprise introducing one or several chemical modifications in the aptamer. Typically, such modifications encompass replacing nucleotide(s) of the aptamer by corresponding chemically-modified nucleotides. The modifications may be performed in order to increase the stability of the aptamers or to introduce chemical moiety enabling functionalization or immobilization on a support.

Further aspects and advantages of the present invention are disclosed in the following experimental section, which should be regarded as illustrative and not limiting the scope of the present application.

### EXAMPLES

### Example 1: Identification of Anti-Fibrinogen Aptamers by the Method of the Invention

1. Material and Method

Oligonucleotide Library

The ssDNA library used to perform SELEX process consisted of a 40-base random region flanked by two constant 18-base primer regions.

Fibrinogen

The protein target was human fibrinogen. Different sources of human fibrinogen were used during Selex process:

Human fibrinogen: Two preparations of human fibrinogen were used prepared as purified composition from human plasma with a purity of 95% and 99.9%, respectively.

Transgenic fibrinogen: Transgenic Fibrinogen was purified from the milk of transgenic cows to 97% purity.

SELEX Protocol

Fibrinogen (97% pure transgenic Fibrinogen for round 1 to 3 and 95% pure plasmatic Fibrinogen for round 4&5 and 99.9% pure plasmatic Fibrinogen for round 6 to 8) was immobilised on an affinity resin, while the amount of target immobilised on the resin continuously decreased from round 1 to 8 (see FIG. 8).

The immobilised target was incubated with the ssDNA library/pool at decreasing concentrations using as selection buffer (50 mM MOPS pH 6.30, 150 mM NaCl, 5 mM MgCl₂) at decreasing incubation time (see table of FIG. 8).

The fibrinogen/ssDNA containing resin was recovered and washed with selection buffer during round 1 & 2 and wash buffer containing 50 mM MOPS pH 6.30, 500 mM NaCl, 5 mM MgCl₂ from round 3 to 8 (see table). After washing, the bound ssDNA was eluted using elution buffer (50 mM Tris-HCl pH 7.40, 200 mM EDTA). Before every round (except the first round) a counter selection step was performed by incubating the ssDNA pool with the affinity resin in order to prevent the enrichment of anti-support aptamers. The parameters of the SELEX protocols are depicted in FIG. 8.

Determination of the Binding Affinity of Aptamers by SPR

The selected aptamer was synthetized with Biotin and a triethylene glycol spacer at the 5′ end of the oligonucleotide. A 1 μM solution of the aptamer was prepared using the SELEX selection buffer. The aptamer solution was heated to 90° C. for 5 min, incubated on ice for 5 min and equilibrated to room temperature for 10 min. The preparation was injected on a streptavidin coated sensor chip SA of Biacore T200 instrument (GE Healthcare) at a flow rate of 10 μl/min for 7 min. Then, different concentrations of the target were injected to the immobilised aptamer at 30 μl/min for 1 minute. After dissociation for 1-2 min a wash step was performed by injecting a suitable wash buffer at 30 μl/min for 1 min. For elution, a suitable elution buffer was injected at 30 μl/min for 1-2 min. Finally the sensor chip was regenerated by injection of 50 mM NaOH at 30 μl/min for 30 sec. During the course of the experiment the response signal was recorded in a sensorgram.

2. Results

The SELEX method of the invention enables to identify 67 anti-fibrinogen aptamer candidates, among which aptamers of SEQ ID NO:1, SEQ ID NO:58, SEQ ID NO:60 and 65 displayed a high affinity for both plasma and transgenic human fibrinogen.

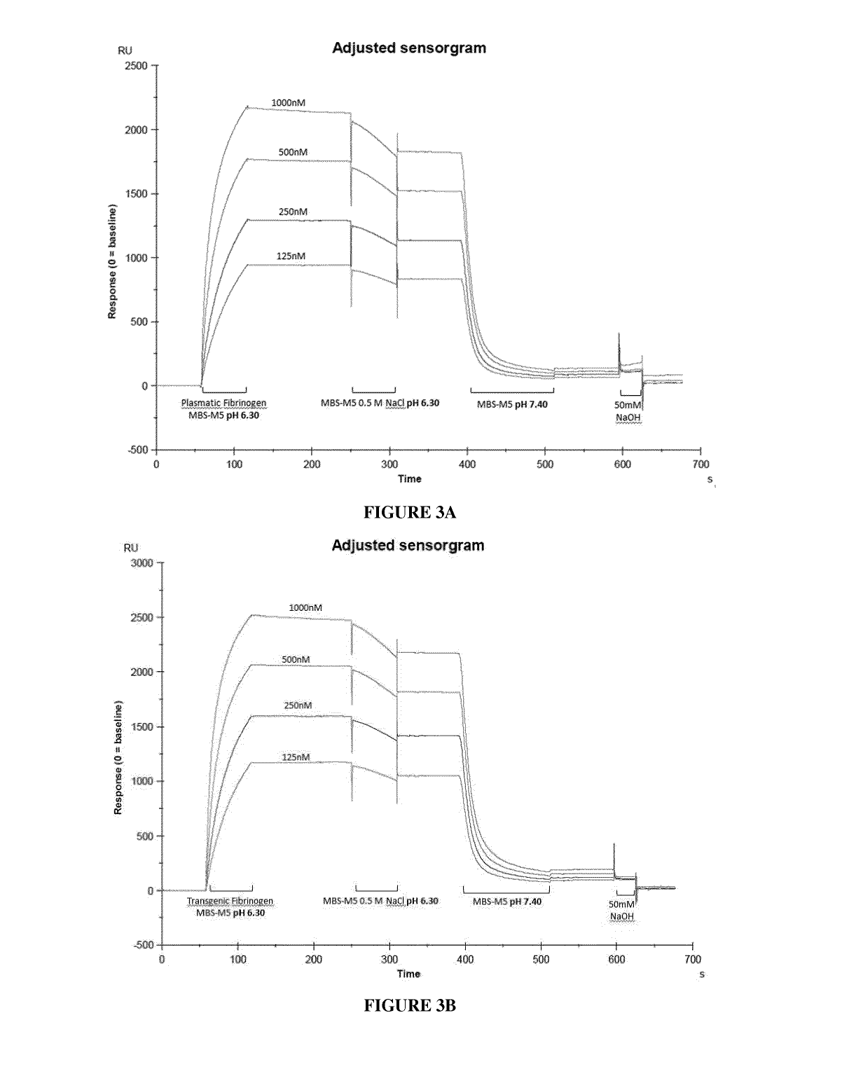

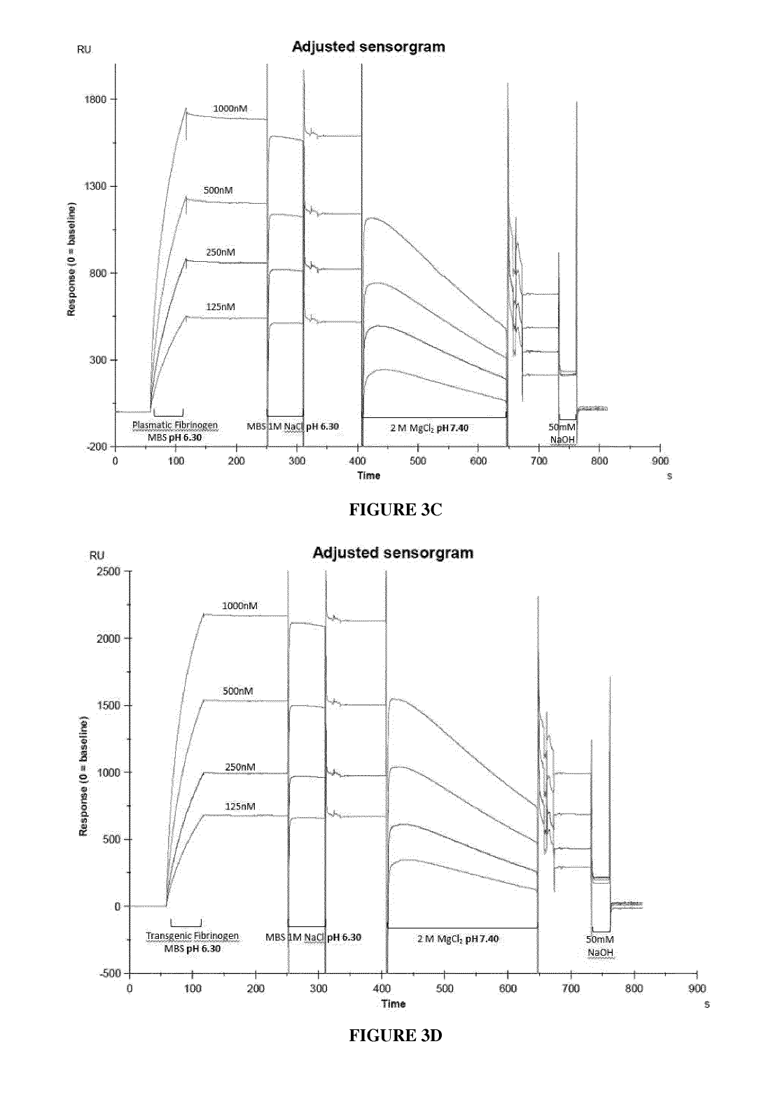

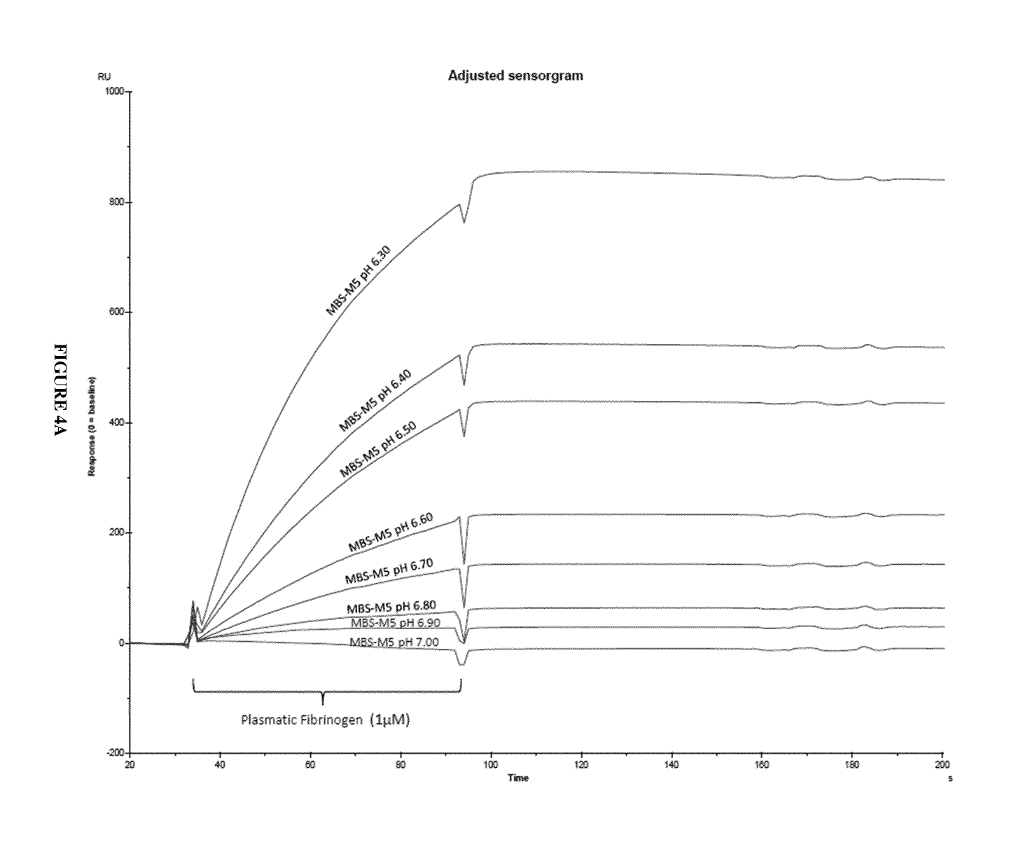

These aptamers were shown to bind human fibrinogen in a pH dependent manner. The core sequences of aptamers SEQ ID NO:1 and SEQ ID NO:58 (namely the minimal sequence binding to fibrinogen) were determined. The aptamer of SEQ ID NO:66 corresponds to the core sequence of aptamer of SEQ ID NO:1. The aptamer of SEQ ID NO:67 is the core sequence of aptamer of SEQ ID NO:58. FIGS. 3A-3D show the binding profile obtained for the core sequences of aptamers of SEQ ID NO:1 and NO:58 by SPR. Aptamers of SEQ ID NO:66 and 67 are able to specifically bind to transgenic fibrinogen and plasma fibrinogen at pH 6.3 in a dose-dependent manner, as evidenced by the increase of the signals when the concentration of fibrinogen was increased. The complex between the aptamers and fibrinogen were not significantly dissociated by the increase of NaCl concentration. On the other hands, the injection of a buffer at pH 7.4 enabled to dissociate the complex between the aptamers and whereby fibrinogen was eluted (FIGS. 3A-3D).

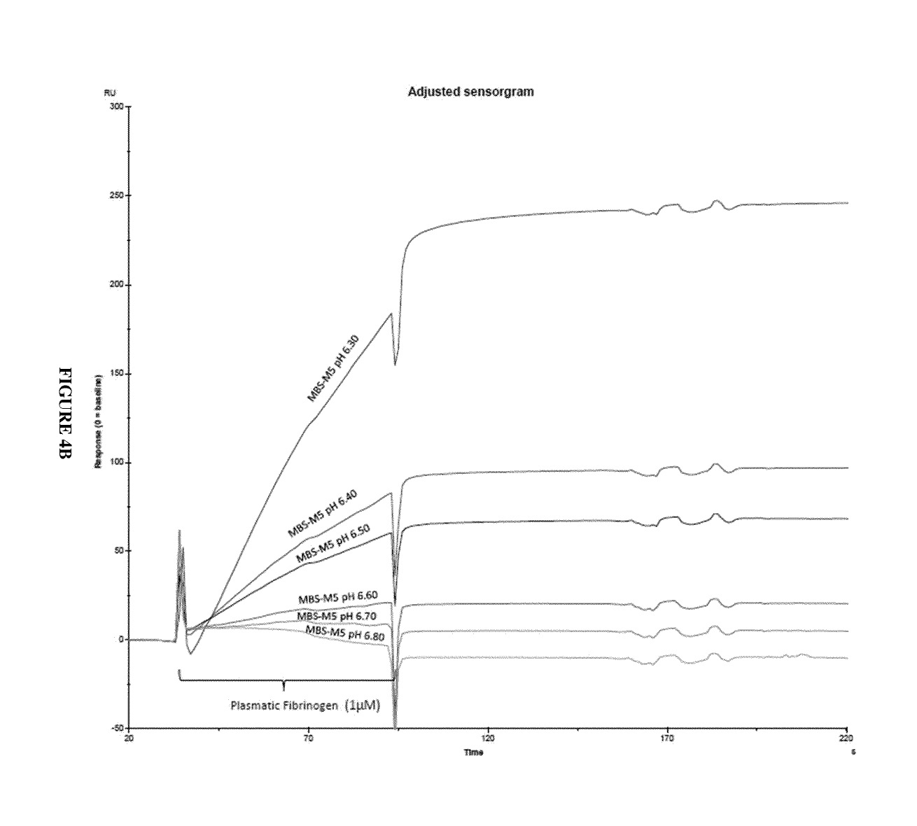

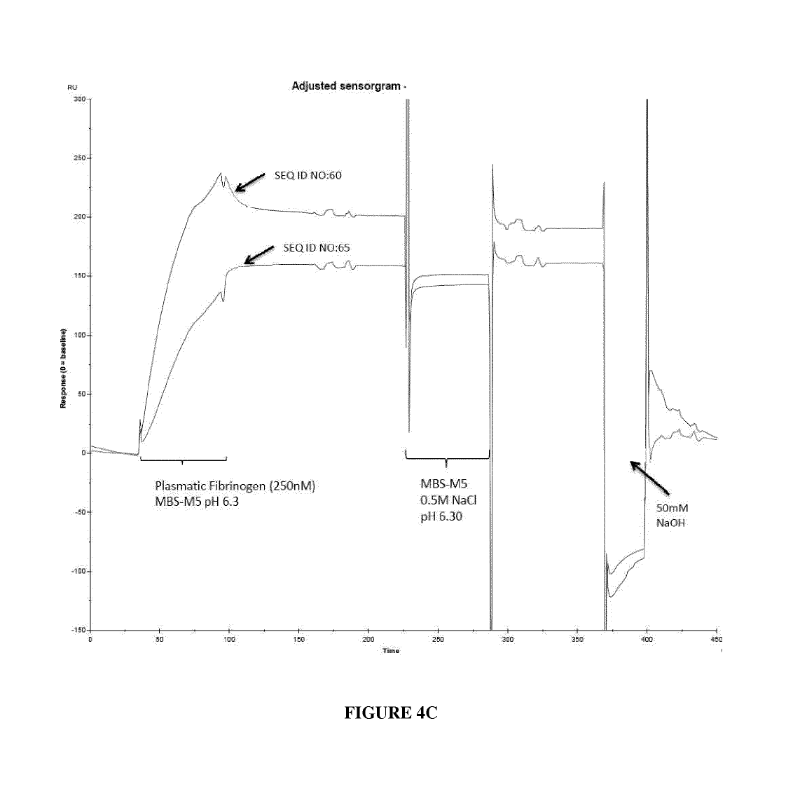

Indeed, the aptamers obtained by the method of the invention bind to fibrinogen in a pH-dependent manner. Such a result is illustrated in FIGS. 4A and 4B for aptamers of SEQ ID NO:66 and SEQ ID NO:67 respectively. The binding level of fibrinogen decreases when pH increased. The highest binding was observed at pH 6.3. The aptamers did not bind to fibrinogen for pH higher than 6.8.

### Example 2: Preparation of Affinity Supports from Aptamers Identified by the Method of the Invention

1. Material and Method

Affinity Supports

Two affinity supports were prepared by grafting aptamers on NHS-activated Sepharose (GE Healthcare). The first affinity support (affinity support no 1) was prepared by grafting aptamers of SEQ ID NO:66 (aptamer A5-1.9) comprising a C6 spacer with a terminal amino group at its 5′ end and an inverted deoxy-thymidine at its 3′ end. The second affinity support (affinity support no 2) was prepared by grafting aptamers of SEQ ID NO:67 (aptamer A5-2.9) comprising a C6 spacer with a terminal amino group at its 5′ end and an inverted deoxy-thymidine at its 3′ end.

1 volume of NHS activated Sepharose gel placed in a column was rinsed with at least 10 volumes of a cold 0.1 M HCl solution, then equilibrated with at least 8 volumes of cold 100 mM acetate pH 4.0 solution.

After a 3 min-2000 g centrifugation, the supernatant is removed and drained gel is resuspended with 2 volumes of aptamer in 100 mM acetate pH 4.0 solution. This suspension is incubated for 2 hours at room temperature under stiffing.

Then, 1 volume of 200 mM Borate pH 9 is added, this suspension is incubated at room temperature under stiffing for 2 h 30.

After a 3 min-2000 g centrifugation, the supernatant is discarded. Drained gel is resuspended in 2 volumes of Tris-HCl 0.1M pH 8.5 solution. Suspension is incubated at +4° C. under stiffing overnight.

After incubation, and a 3 min-2000 g centrifugation, the supernatant is discarded. The gel is alternatively washed with 2 volumes of Sodium acetate 0.1M+NaCl 0.5M pH 4.2 and 2 volumes of a Tris-HCl 0.1M pH 8.5 solution. This washing cycle is repeated once.

After a 3 min-2000 g centrifugation supernatant is removed. The drained gel is resuspended in 2 volumes of equilibration buffer.

| | | Affinity support | | Affinity support | |
|---|---|---|---|---|---|
| | | no1 grafted with | | no2 grafted with | |
| | | aptamer moieties of | | aptamer moieties of | |
| | | SEQ ID NO: 66 | | SEQ ID NO: 67 | |
| Fibrinogen | Quantity of | 4.2 | mg | 2.5 | mg |
| purification | aptamer used | | | | |
| from Plasma | for grafting | | | | |
| | Volume of | 0.5 | mL | 0.5 | mL |
| | grafted gel | | | | |
| Fibrinogen | Quantity of | 174 | mg | 209 | mg |
| purification | aptamer used | | | | |
| from semi | for grafting | | | | |
| purified | Volume of | 24 | mL | 42 | mL |
| fibrinogen | grafted gel | | | | |
| product | | | | | |

### Example 3: Purification of Fibrinogen from Semi Purified Fibrinogen Solution on the Affinity Support of Examples 2

1. Material and Method

Conditions of the Affinity Chromatography

Affinity support no 1: Thawed semi purified fibrinogen solution (IP1: Fibrinogen Intermediate Product 1) obtained from human plasma was diluted 10 times in the binding buffer and was pH adjusted to 6.3. Diluted IP1 was subjected to a chromatography steps on support no 1. This step was repeated once to obtain enough fibrinogen quantity for ultrafiltration step.

Affinity support no 2: Thawed semi purified fibrinogen solution (IP1: Fibrinogen intermediate product 1) obtained from human plasma was diluted 10 times in the binding buffer and pH was adjusted to 6.3. Diluted IP1 was subjected to a chromatography steps on support no 2. This step was repeated once to obtain enough fibrinogen quantity for ultrafiltration step.

The conditions of the affinity chromatography are summarized for each affinity support:

| | Affinity support | Affinity support |
|---|---|---|
| | no1 grafted with | no2 grafted with |
| | aptamer moieties of | aptamer moieties of |
| | SEQ ID NO: 66 (A5-1.9) | SEQ ID NO: 67 (A5-2.9) |
| Binding buffer | MOPS 50 mM, MgCl₂ | MOPS 50 mM, NaCl |
| | 5 mM, NaCl 150 mM, | 150 mM, pH 6.3 |
| | pH 6.3 | |
| Washing buffer | None | MOPS 50 mM, NaCl |
| | | 2M, pH 7.4 |
| Elution buffer | MOPS 50 mM, NaCl | MOPS 50 mM, MgCl₂ |
| | 150 mM, pH 7.4 | 2M, pH 7.4 |

For each affinity support, fibrinogen was eluted in mild conditions by modification of the buffer composition. For both chromatography on Affinity support no 1 and n° 2: 2 eluate fractions were generated and pooled for ultrafiltration step.

Conditions of the Ultrafiltration

For each affinity support, pool of eluate fractions were subjected to an ultrafiltration 100 kDa in order to concentrate Fibrinogen and to formulate in sodium citrate 10 mM, arginine 20 g/L at pH 7.4.

Analytical Methods

| Proteins | Titration methods |
|---|---|
| Fibronectin, antigenic Fibrinogen | Nephelometry |
| Factor II, Factor XI, | Elisa |
| Factor XIII, Plasminogen | |
| Fibrinogen clotting activity | Coagulation assay |
| | (von Clauss method) |

2. Results

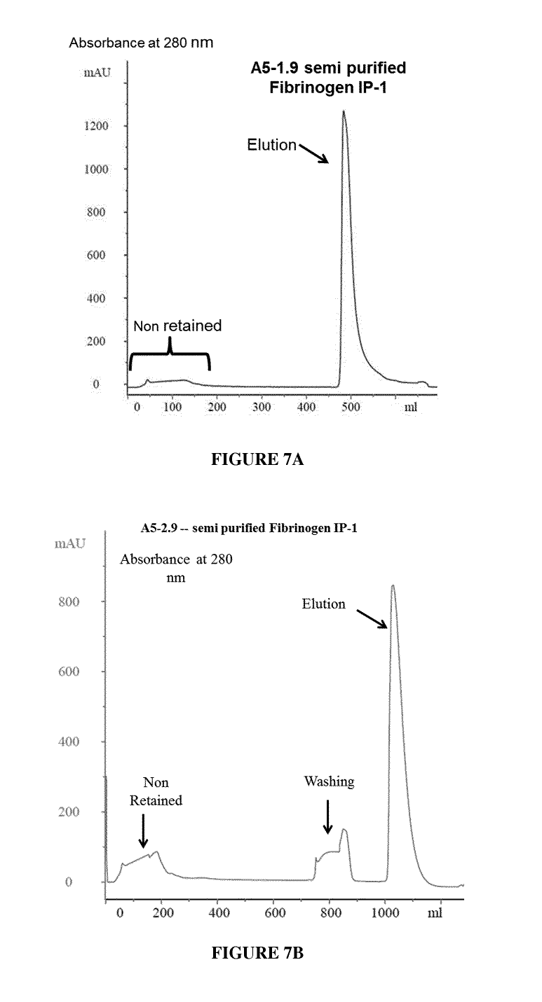

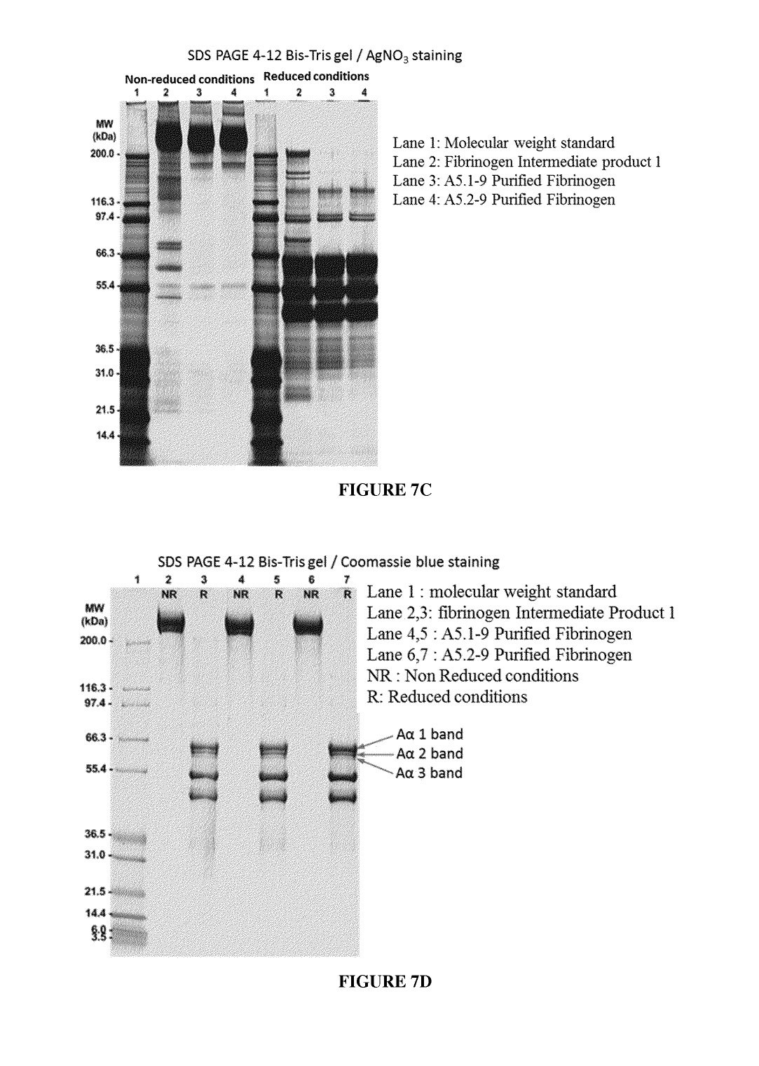

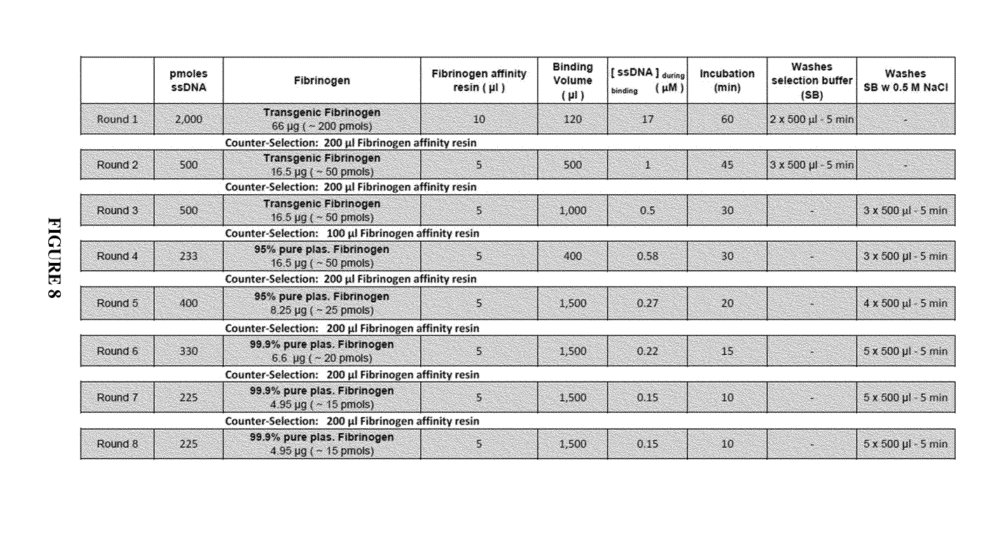

The results are shown in FIGS. 7A-7B and 7C-7D. FIGS. 7A and 7B show the chromatography profile obtained for the fibrinogen purification from semi purified fibrinogen solution on the affinity support no 1 and n° 2 respectively. Fibrinogen was eluted by increasing the pH to 7.4 and by adding MgCl₂ for affinity support no 2 and by suppressing Mg²⁺ for affinity support no 1. The electrophoresis analysis of the fractions obtained by chromatography (FIGS. 7C and 7D) showed that contaminants present in the loaded material (IP1) are drastically removed with almost only Fibrinogen visible in the eluate. Additionally reducing conditions shows that Fibrinogen in the eluate is in a native form with no visible degradation (Aα1 is the most important band of Aα bands)

Yields and Fibrinogen Concentration Obtained are Summarized in the Table Below:

| | Affinity | Affinity |
|---|---|---|
| | support no1 | support no2 |
| Chromatography yield (%) | 51 | 71 |
| Concentration of antigenic fibrinogen | 13.1 | 14.2 |
| obtained after ultrafiltration (mg/ml) | | |

Active Fibrinogen is demonstrated by a ratio between coagulant Fibrinogen and antigenic Fibrinogen close to 1. Analysis on the concentrated and formulated fibrinogen prepared with both affinity supports are detailed in the following table:

| | clotting activity | Fibrinogen/ratio |
|---|---|---|
| | Fibrinogen g/L | clotting/antigenic |
| Starting material | 17.6 | 1.17 |
| (IP1 fibrinogen) | | |
| Purified Fibrinogen | 13.9 | 1.06 |
| concentrate - Support no1 | | |
| Purified Fibrinogen | 14.5 | 1.02 |
| concentrate - Support no2 | | |

For both purified Fibrinogen, the ratio between clotting and antigenic fibrinogen was about 1.0 for both aptamers. The soft chromatography conditions allowed the preparation of a purified fibrinogen with preserved activity.

The table hereunder shows the titration of the contaminant proteins in the starting material and the purified fibrinogen fraction obtained with the affinity support of the invention:

| | semi purified | | | | | | | |
|---|---|---|---|---|---|---|---|---|
| | Fibrinogen | | | | | | | |
| | (starting | | | | | | | |
| Contaminant | composition) | | Affinity support no1 | | | Affinity support no2 | | |
| proteins | Concentration | | Concentration | | Removal | Concentration | | Removal |
| Fibronectin | 0.55 | g/E | 0.02 | g/L | 96.3% | 0.02 | g/L | 95.1% |
| Factor II | 0.13 | mUI/mL | 0.03 | mUI/mL | 70.8% | 0.04 | mUI/mL | 66.3% |
| Factor XI | 21.0 | mUI/mL | 2.8 | mUVmL | 83.8% | 3.6 | mUI/mL | 80.8% |
| Factor XIII | 10000 | mUI/mL | 10 | mUI/mL | 99.9% | 42 | mUI/mL | 99.5% |
| Plasminogen | 56 | μg/mL | 0.21 | μg/mL | 99.5% | 0.21 | μg/mL | 99.6% |

A good elimination of contaminants proteins is obtained with a removal comprised from 65% to over than 99% from starting material.

Chromatography conditions allowed the removal of more than 99.5% of initial plasminogen, which is one of the most problematic contaminant with regards to Fibrinogen stability.

The aptamers identified by the SELEX of the invention are suitable for use as affinity ligand in the purification of fibrinogen by chromatography. Noteworthy, the aptamers identified by the process of the invention enables the selective binding and then the elution of fibrinogen in mild and non-denaturing conditions.

### Example 4: Purification of Fibrinogen by Chromatography from Plasma

Affinity support no 1: The Plasma was thawed, filtrated 0.45 μm, diluted 10 times in the binding buffer and then pH adjusted to 6.3. Diluted solution was subjected to a chromatography steps on support no 1.

Affinity support no 2: The Plasma was thawed, filtrated 0.45 μm, diluted 10 times in the binding buffer and then pH adjusted to 6.3. Diluted solution was subjected to a chromatography steps on support no 2.

The conditions of the affinity are summarized, for each affinity support, in the table below:

| | Affinity support | Affinity support |
|---|---|---|
| | no1 grafted with | no2 grafted with |
| | aptamer moieties of | aptamer moieties of |
| | SEQ ID NO: 66 (A5-1.9) | SEQ ID NO: 67 (A5-2.9) |
| Binding buffer | MOPS 50 mM, MgCl₂ | MOPS 50 mM, NaCl |
| | 5 mM, NaCl 150 mM, | 150 mM, pH 6.3 |
| | pH 6.3 | |
| Washing buffer | return to baseline with | MOPS 50 mM, NaCl 2M, |
| | the binding buffer | pH 7.4 |
| Elution buffer | MOPS 50 mM, NaCl | MOPS 50 mM, MgCl₂ |
| | 150 mM, pH 7.4 | 2M, pH 7.4 |
| Regeneration | MOPS 50 mM, MgCl₂ | same as the elution |
| buffer | 2M, pH 7.4 | buffer |

For each affinity support, fibrinogen was eluted in mild conditions by modification of the buffer composition.

2. Results

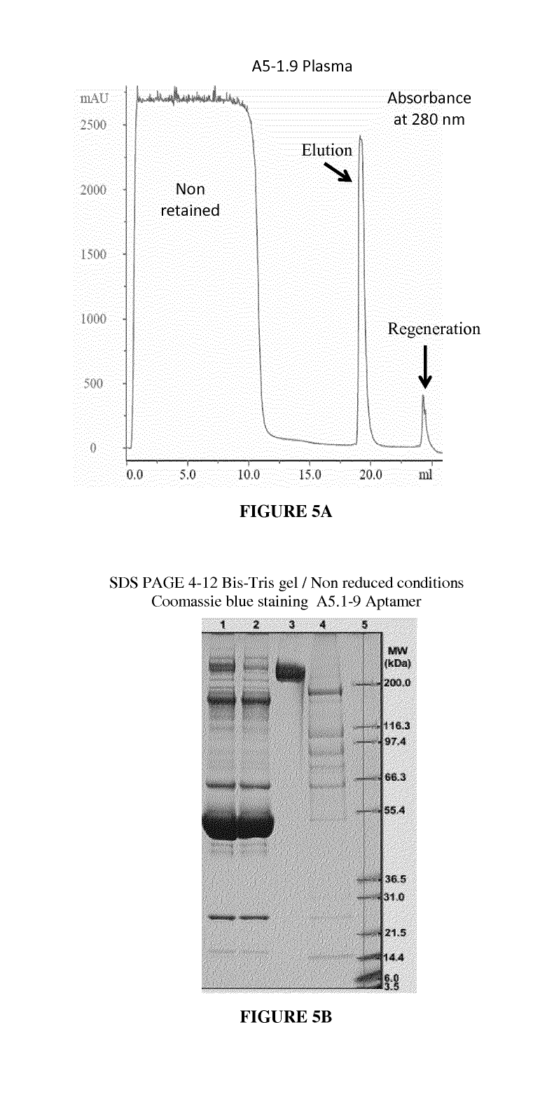

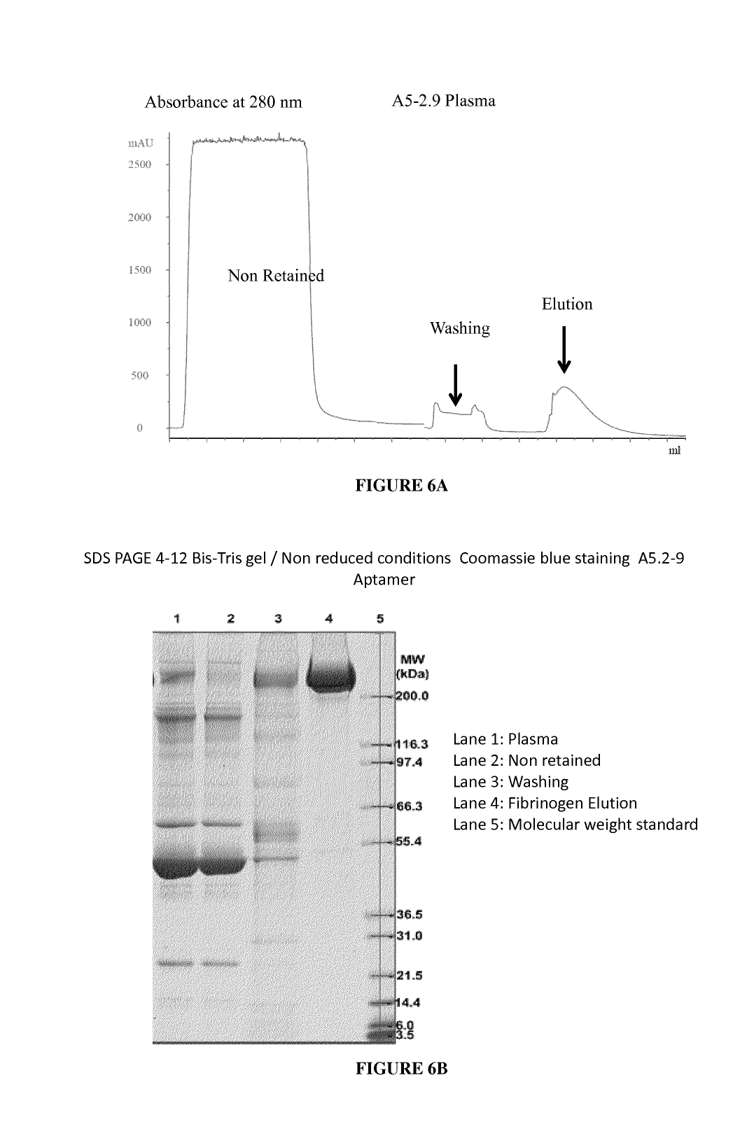

The results are shown in FIGS. 5A-5B and 6A-6B. FIGS. 5A and 6A show the chromatography profile obtained for the purification of fibrinogen from plasma on the affinity support no 1 and n° 2 respectively. Noteworthy, most of the contaminant proteins were not retained on the stationary phase whereas fibrinogen bound to the support. Fibrinogen was eluted by increasing the pH to 7.4 and by adding 2 M MgCl₂ for affinity support no 2 and by suppressing Mg²⁺ for affinity support no 1. The electrophoresis analysis of the fractions obtained by chromatography (FIG. 5B and FIG. 6B) showed that fibrinogen was mostly present in the elution fraction whereas contaminant proteins were present in the non-retained fraction, in the washing fraction or the regeneration fraction. Indeed, the elution fractions migrated as single band. The relative purity (determined by SDS PAGE) of the eluate fibrinogen fractions was greater than 95%.

Such results demonstrate that the aptamers of the invention are particularly suitable for a use as affinity ligands in the purification of fibrinogen from complex starting compositions.

### Example 5: Optimization of the Core Sequence of SEQ ID NO:66

1. Material and Method

Preparation of Variants of SEQ ID NO:66:

In the first round, 28 variants were designed by systematically removing 2 consecutive nucleotides per variant (½, ¾, ⅚ etc.). In the next round combinations of deletions that did not lead to a loss of affinity were combined.

Competitive Binding Assay

In order to compare the affinity of the designed variants to the parental aptamer a competition assay was performed. First, a 1 μM solution of the aptamer SEQ ID NO:66 was prepared using the binding buffer. The aptamer solution was heated to 90° C. for 5 min, incubated on ice for 5 min and equilibrated to room temperature for 10 min. The preparation was injected on a streptavidin coated sensor chip SA of Biacore T200 instrument (GE Healthcare) at a flow rate of 10 μl/min for 7 min. Then, mixtures containing one variant (2 μM) and human plasmatic fibrinogen (0.4 μM) were injected to the immobilised aptamer at 30 μl/min for 1 minute. The response obtained for the different fragment/fibrinogen mixtures was compared.

2. Results

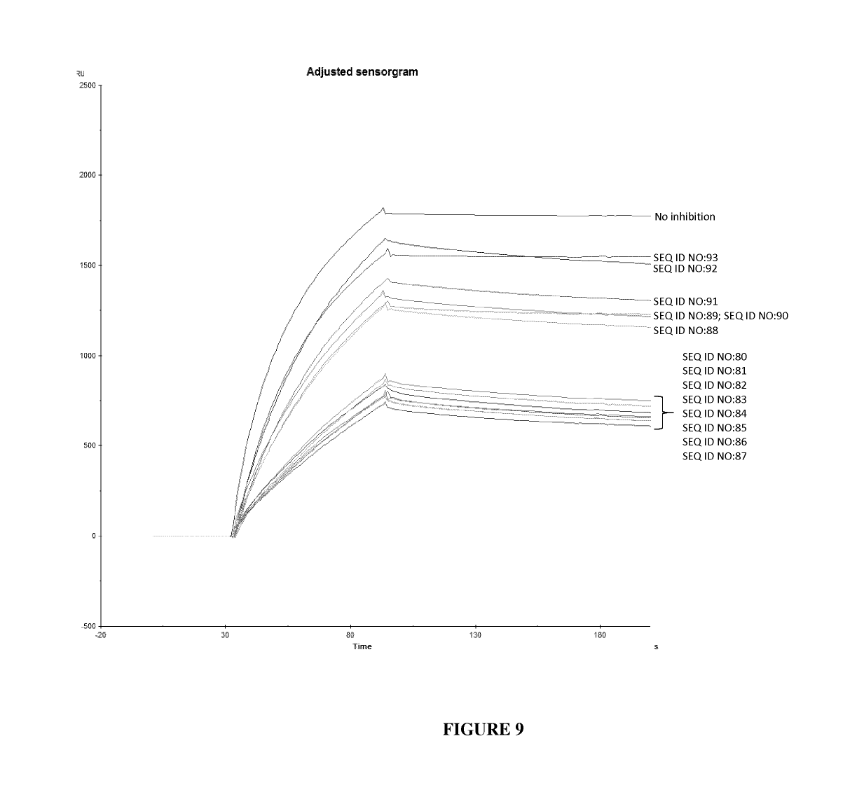

As shown in FIG. 9, sequence variants SEQ ID NO:80-93 show a significant inhibition of the binding signal and therefore possess a considerable affinity for fibrinogen. The highest affinity was observed for variants SEQ ID NO:80-87 containing deletions at 01/02, at positions 01/02/19/20/21, at positions 01/02/14/15/16/20/21/22, at positions 01/02/18/19/20/21, at positions 01/02/18/19/20, at positions 01/02/15/16/20/21, and at positions 01/02/19/20, respectively.

### Example 6: Stability of the Purified Fibrinogen Obtained by the Method of the Invention

A liquid composition containing plasmatic fibrinogen was purified from semi-purified fibrinogen as described in Example 3, namely by an affinity chromatography step followed by an ultrafiltration and formulation of the elution fraction containing fibrinogen. The affinity ligand was aptamer A-5.1.9 (SEQ ID NO:66). The binding buffer and the elution buffer were as described in example 3. The resulting composition was an aqueous solution of fibrinogen containing sodium citrate at 8.5 mM and arginine HCl 100 mM, at pH 6.9 and with an osmolality of 206 mOsm/kg.

The stability of the liquid composition was tested under the following conditions:

The liquid solution was packaged in a vial, under air, and maintained without stiffing, in a chamber with a controlled temperature at 5° C. during one month.

Several parameters enabling to assess the stability of the liquid composition were determined before (TO) and after (T1M) the stability test such as the percentages of Aα1 and Aα2 determined by SDS PAGE in reduced conditions, and the antigenic/clotting activity ratio.

Results

The visual aspect and the turbidity of the liquid composition of fibrinogen did not significantly change before and after the stability test. No significant variation in the pH and in the osmolality of the solution was observed. The electrophoresis gels obtained by SDS-PAGE under non-reduced condition showed one single band with 100% of intensity, at TO and TIM.

The tables hereunder show the results for the other tested parameters:

SDS PAGE analyses under reduced conditions:

| | | T0 | T1 M |
|---|---|---|---|
| | MW kDa/band | Intensity % | Intensity % |
| Aα1 | 64 | 23.7 | 25.7 |
| Aα2 | 62 | 11.7 | 10.4 |
| Aα3 | 60 | 3.7 | 2.3 |
| Σα | — | 39.1 | 38.4 |
| B | 54 | 31.2 | 31.9 |
| γ′ | 50 | 6 | 5.6 |
| γ | 48 | 23.7 | 24.1 |
| Σγ | — | 29.7 | 29.7 |

Noteworthy, there was no significant variation in the percentage of Aα1 and Aα3 between TO and T1M, which illustrated the absence of substantial degradation of the fibrinogen.

Antigenic Activity/Clotting Activity

| | Clotting activity | Antigenic activity | clotting/antigenic |
|---|---|---|---|
| — | (g/L) | (g/L) | ratio |
| T0 | 15.1 | 14.2 | 1.1 |
| T1 M | 14.4 | 13.4 | 1.1 |

The clotting/antigenic ratio was constant during the stability test.

All these results demonstrate that the liquid composition prepared with fibrinogen obtained by the method of the invention was stable under storage, at 5° C.

### TABLE OF SEQUENCES

| NO of SEQ ID | Description |
|---|---|
| 1-57 | Aptamers of the first subgroup |
| 58-65 | Aptamers of the second subgroup |
| 66 | Core sequence of the aptamer of |
| | SEQ ID NO: 1 (A.5.1.9) |
| 67 | Core sequence of the aptamer of |
| | SEQ ID NO: 58 (A.5.2.9) |
| 68-74 | Central regions of SEQ ID NO: 59-65 |
| 75 | First primer sequence |
| 76 | Second primer sequence |
| 77-79 | Nucleotide moieties present in the |
| | consensus sequence of the second |
| | subgroup of aptamers |
| 80-93 | Variants of the aptamer of SEQ ID NO: 66 |
| 94 | Central region of SEQ ID NO: 1 |
| 95 | Central region of SEQ ID NO: 58 |

1.-16. (canceled)

17. An aptamer which specifically binds to fibrinogen in a pH-dependent manner.

18. The aptamer of claim 17 which does not bind to fibrinogen at a pH higher than 7.0 but specifically binds to fibrinogen at an acid pH value.

19. The aptamer of claim 18, wherein the acid pH value is 6.0 to 6.6.

20. An aptamer capable of specifically binding to fibrinogen, wherein:
the aptamer comprises a polynucleotide having at least 70% of sequence identity with the nucleotide sequence of SEQ ID No 66, or
the aptamer comprises the nucleotide moiety of formula (II):
5′-[SEQ ID NO: 79]-[X1]SEQ ID NO: 77]-[X2]- [SEQ
ID NO: 78]-3′ (III)
wherein:
[X2] and [X1] are independently a nucleotide or an oligonucleotide of 2 to 5 nucleotides in length,
[SEQ ID NO:77] is an oligonucleotide of SEQ ID NO:77 (namely GTTGGTAGGG),
[SEQ ID NO:78] is an oligonucleotide of SEQ ID NO:78 (namely GGTGTAT), and
[SEQ ID NO:79] is an oligonucleotide of SEQ ID NO:79 (namely TGT).

21. The aptamer of claim 20, wherein the aptamer has at least 70% sequence identity with SEQ ID NO:66, SEQ ID NO:67, SEQ ID NO:68, SEQ ID NO:69, SEQ ID NO:70, SEQ ID NO:71, SEQ ID NO:72, SEQ ID NO:73, or SEQ ID NO:74.

22. The aptamer of claim 20, wherein the aptamer is of formula (I)
5′-[NUC1]m-[CENTRAL]-[NUC2]n-3′
Wherein
n and m are integers independently selected from 0 and 1,
[NUC1] is a polynucleotide comprising from 2 to 40 nucleotides,
[NUC2] is a polynucleotide comprising from 2 to 40 nucleotides, and
[CENTRAL] is a polynucleotide having at least 70% of sequence identity with SEQ ID NO:68, SEQ ID NO:69, SEQ ID NO:70, SEQ ID NO:71, SEQ ID NO:72, SEQ ID NO:73 SEQ ID NO:74, SEQ ID NO:94, or SEQ ID NO:95.

23. The aptamer of claim 20, wherein the aptamer comprises SEQ ID NO:66, or differs from SEQ ID NO:66 by 1 to 14 nucleotide modifications at nucleotide positions selected from 1, 2, 11-25, 32-35, 42, 45-47, 50 and 54-58, wherein the numbering refers to nucleotide numbering in SEQ ID NO:66.

24. The aptamer of claim 23, wherein the aptamer comprises SEQ ID NO:66, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, or SEQ ID NO:93.

25. The aptamer of claim 20, wherein the aptamer comprises at least 70% sequence identity with a nucleotide sequence selected from the group consisting of SEQ ID NO:1 to SEQ ID NO:67.

26. The aptamer of claim 20, wherein the aptamer specifically binds to human plasma fibrinogen or recombinant human fibrinogen.

27. An affinity ligand capable of specifically binding to fibrinogen, comprising an aptamer as defined in claim 20 and at least one moiety selected from a means of detection and a means of immobilization onto a support.

28. A solid affinity support comprising thereon a plurality of affinity ligands as defined in claim 27.

29. A method for preparing a purified fibrinogen composition from a starting fibrinogen-containing composition, comprising:
a) contacting said starting composition with an affinity support as defined in claim 28, in conditions suitable to form a complex between (i) the affinity ligands immobilized on said support and (ii) fibrinogen;
b) releasing fibrinogen from said complex; and
c) recovering a purified fibrinogen composition.

30. The method of claim 29 wherein step a) is performed at a pH lower than 7.0, and step b) is performed at a pH above 7.0.

31. The method of claim 30, wherein step a) is performed at a pH of 6.0 to 6.6, and step b) is performed at a pH of 7.2 to 7.6.

32. The method of claim 29, wherein steps a), b), and c) are performed by chromatography technology.

33. A method of purifying fibrinogen, detecting fibrinogen, blood plasma fractionation, or preparing a composition comprising fibrinogen that is stable in liquid form, comprising using the affinity ligand of claim 27.

34. A blood plasma fractionation process comprising:
(a) an affinity chromatography step to recover fibrinogen wherein the affinity ligand is an aptamer which specifically binds to fibrinogen as defined in claim 20,
(b) an affinity chromatography step to recover immunoglobulins (Ig) wherein the affinity ligand specifically binds to immunoglobulins, and
(c) optionally a purification step of albumin,
wherein steps (a), (b) and (c) can be performed in any order.

35. A composition comprising fibrinogen which is stable in liquid form, which is obtainable by a method comprising the steps of:
providing a blood plasma or a cryosupernatant fraction of blood plasma,
purifying said blood plasma or said cryosupernatant fraction of blood plasma by separation on affinity chromatography gel using an affinity ligand selected from aptamers as defined in claim 20,
collecting the purified adsorbed fraction comprising fibrinogen, and
optionally, adding pharmaceutically acceptable excipients, preferably arginine and/or citrate such as citrate salt.

36. A method for preparing a purified fibrinogen composition from a starting fibrinogen-containing composition comprising:
a) contacting said starting composition with an affinity support on which aptamers as defined in claim 17 are immobilized, in conditions suitable to form a complex between (i) said aptamers immobilized on the support and (ii) fibrinogen;
b) releasing fibrinogen from said complex; and
c) recovering a purified fibrinogen composition.


SEQUENCE LISTING (95 sequences)

SEQ ID NO: 1 - DNA, 76 bp, Artificial
  Aptamer
gggtcaatgccaggtctcggacctggaatccgccacccgcattagaaccagggttgacatcggctcgcaagcagtc

SEQ ID NO: 2 - DNA, 76 bp, Artificial
  Aptamer
gggtcaatgccaggtctcggacctggaatccgccacccgcactagaaccagggttgacatcggctcgcaagcagtc

SEQ ID NO: 3 - DNA, 75 bp, Artificial
  Aptamer
gggtcaatgccaggtctcggacctggaatccgccacccgcattagaaccagggttgacatcggctcgcagcagtc

SEQ ID NO: 4 - DNA, 74 bp, Artificial
  Aptamer
gggtcaatgccaggtctcggacctggaatccgccacccgcattagaaccagggttgacatcggctcgcgcagtc

SEQ ID NO: 5 - DNA, 75 bp, Artificial
  Aptamer
gggtcaatgccaggtctcggacctggaatccgccacccgcattagaaccagggttgacatcggctcgcaagcagc

SEQ ID NO: 6 - DNA, 76 bp, Artificial
  Aptamer
gggtcaatgccaggtctcggacctggaatccgccacccgcattagaaccagggttgacgtcggctcgcaagcagtc

SEQ ID NO: 7 - DNA, 76 bp, Artificial
  Aptamer
gggtcaatgccaggtctcagacctggaatccgccacccgcattagaaccagggttgacatcggctcgcaagcagtc

SEQ ID NO: 8 - DNA, 76 bp, Artificial
  Aptamer
gggtcaatgccaggtctcggacctggaatccgccacccgcattaaaaccagggttgacatcggctcgcaagcagtc

SEQ ID NO: 9 - DNA, 76 bp, Artificial
  Aptamer
gggtcaatgccaggtctcggacctggaatccgtcacccgcattagaaccagggttgacatcggctcgcaagcagtc

SEQ ID NO: 10 - DNA, 76 bp, Artificial
  Aptamer
gggtcaatgccaggtctcggacctggaatccgccacccgcattagaaccagggttgacatcggctcgcaagcggtc

SEQ ID NO: 11 - DNA, 76 bp, Artificial
  Aptamer
gggtcaatgccaggtctcggacccggaatccgccacccgcattagaaccagggttgacatcggctcgcaagcagtc

SEQ ID NO: 12 - DNA, 76 bp, Artificial
  Aptamer
gggtcaatgccaggtctcggacctggaatccgccacccgcattagaaccagggttgacatcgactcgcaagcagtc

SEQ ID NO: 13 - DNA, 75 bp, Artificial
  Aptamer
gggtcaatgccaggtctcggacctggaatccgccacccgcactagaaccagggttgacatcggctcgcaagcatc

SEQ ID NO: 14 - DNA, 75 bp, Artificial
  Aptamer
gggtcaatgccaggtctcggacctggaatccgccacccgcactagaaccagggttgacatcggctcgcaagcgtc

SEQ ID NO: 15 - DNA, 76 bp, Artificial
  Aptamer
gggtcaatgccaggtctcggacccggaatccgccacccgcactagaaccagggttgacatcggctcgcaagcagtc

SEQ ID NO: 16 - DNA, 76 bp, Artificial
  Aptamer
gggtcaatgccaggtctcggaccaggaatccgccacccgcactagaaccagggttgacatcggctcgcaagcagtc

SEQ ID NO: 17 - DNA, 75 bp, Artificial
  Aptamer
gggtcaatgccaggtctcggacctggaatccgccacccgcactagaaccagggttgacatcggctgcaagcagtc

SEQ ID NO: 18 - DNA, 76 bp, Artificial
  Aptamer
gggtcaatgccaggtctcggacctggaatccgccacccgcactagagccagggttgacatcggctcgcaagcagtc

SEQ ID NO: 19 - DNA, 76 bp, Artificial
  Aptamer
gggtcaatgccaggtctcggacctggaatccgccacccgcactagaaccagggttgacatcggctcacaagcagtc

SEQ ID NO: 20 - DNA, 75 bp, Artificial
  Aptamer
gggtcaatgccaggtctcggacctggaatccgccacccgcactagaaccagggttgacatcggctccaagcagtc

SEQ ID NO: 21 - DNA, 74 bp, Artificial
  Aptamer
gggtcaatgccaggtctcggacctggaatccgccacccgcactagaaccagggttgacatcggctcaagcagtc

SEQ ID NO: 22 - DNA, 76 bp, Artificial
  Aptamer
gggtcaatgccaggtctcggacctggaatccgccacccgcactagaaccagggttgacatcggctcggaagcagtc

SEQ ID NO: 23 - DNA, 75 bp, Artificial
  Aptamer
gggtcaatgccaggtctcggacctggaatccgccacccgcactagaaccagggttgacatcggctcgaagcagtc

SEQ ID NO: 24 - DNA, 75 bp, Artificial
  Aptamer
gggtcaatgccaggtctcggacctggaatccgccacccgcactagaaccagggttgacatcggctcgcagcagtc

SEQ ID NO: 25 - DNA, 74 bp, Artificial
  Aptamer
gggtcaatgccaggtctcggacctggaatccgccacccgcactagaaccagggttgacatcggctcgcgcagtc

SEQ ID NO: 26 - DNA, 76 bp, Artificial
  Aptamer
gggtcaatgccaggtctcggacctggaatccgccacccgcactagaaccagggttgacatgggctcgcaagcagtc

SEQ ID NO: 27 - DNA, 74 bp, Artificial
  Aptamer
gggtcaatgccaggtctcggacctggaatccgccacccgcactagaaccagggttgacatcggctcgcacagtc

SEQ ID NO: 28 - DNA, 75 bp, Artificial
  Aptamer
gggtcaatgccaggtctcggacctggaatccgccacccgcactagaaccagggttgacatcggctcgcaacagtc

SEQ ID NO: 29 - DNA, 76 bp, Artificial
  Aptamer
gggtcaatgccaggtctcggacctggaatccgccacccgcactagaaccagggttgacatcagctcgcaagcagtc

SEQ ID NO: 30 - DNA, 76 bp, Artificial
  Aptamer
gggtcaatgccaggtctcggacctggaatccgccacccgcactagaaccagggctgacatcggctcgcaagcagtc

SEQ ID NO: 31 - DNA, 76 bp, Artificial
  Aptamer
gggtcaatgccaggtctcggacctggaatccgccacccgcattagaaccagggctgacatcggctcgcaagcagtc

SEQ ID NO: 32 - DNA, 76 bp, Artificial
  Aptamer
gggtcaatgccaggtctcggacctggaatccgccacccgcactagaaccagggttggcatcggctcgcaagcagtc

SEQ ID NO: 33 - DNA, 76 bp, Artificial
  Aptamer
gggtcaatgccaggtctcggacctggaatccgccacccgcattagaaccagggttggcatcggctcgcaagcagtc

SEQ ID NO: 34 - DNA, 76 bp, Artificial
  Aptamer
gggtcaatgccaggtctcggacctgggatccgccacccgcactagaaccagggttgacatcggctcgcaagcagtc

SEQ ID NO: 35 - DNA, 76 bp, Artificial
  Aptamer
gggtcaatgccaggtctcggacctgggatccgccacccgcattagaaccagggttgacatcggctcgcaagcagtc

SEQ ID NO: 36 - DNA, 76 bp, Artificial
  Aptamer
gggtcaatgccaggtctcggacctggaatccgccacccgcactagaaccagggttgacatcgggtcgcaagcagtc

SEQ ID NO: 37 - DNA, 76 bp, Artificial
  Aptamer
gggtcaatgccaggtctcggacctggaatccgccacccgcattagaaccagggttgacatcgggtcgcaagcagtc

SEQ ID NO: 38 - DNA, 76 bp, Artificial
  Aptamer
gggtcaatgccaggtctcggacctggaatccgccacccgcattagaaccagggtcgacatcggctcgcaagcagtc

SEQ ID NO: 39 - DNA, 76 bp, Artificial
  Aptamer
gggtcaatgccaggtctcggacctggaatccgccacccgcactagaaccagggtcgacatcggctcgcaagcagtc

SEQ ID NO: 40 - DNA, 76 bp, Artificial
  Aptamer
gggtcaatgccaggtctcgggcctggaatccgccacccgcattagaaccagggttgacatcggctcgcaagcagtc

SEQ ID NO: 41 - DNA, 76 bp, Artificial
  Aptamer
gggtcaatgccaggtctcgggcctggaatccgccacccgcactagaaccagggttgacatcggctcgcaagcagtc

SEQ ID NO: 42 - DNA, 76 bp, Artificial
  Aptamer
gggtcaatgccaggtctcggacctggaatccgccgcccgcattagaaccagggttgacatcggctcgcaagcagtc

SEQ ID NO: 43 - DNA, 76 bp, Artificial
  Aptamer
gggtcaatgccaggtctcggacctggaatccgccgcccgcactagaaccagggttgacatcggctcgcaagcagtc

SEQ ID NO: 44 - DNA, 76 bp, Artificial
  Aptamer
gggtcaatgccaggtctcggacctggaatccgcctcccgcattagaaccagggttgacatcggctcgcaagcagtc

SEQ ID NO: 45 - DNA, 76 bp, Artificial
  Aptamer
gggtcaatgccaggtctcggacctggaatccgccacccgcattagaaccagggttgacatcggctggcaagcagtc

SEQ ID NO: 46 - DNA, 74 bp, Artificial
  Aptamer
gggtcaatgccaggtctcggacctggaatccgccacccgcattagaaccagggttgacatcggctcaagcagtc

SEQ ID NO: 47 - DNA, 76 bp, Artificial
  Aptamer
gggtcaatgccaggtctcggacctggaatccgccacccgcattagaaccagggttgacatcggctcacaagcagtc

SEQ ID NO: 48 - DNA, 75 bp, Artificial
  Aptamer
gggtcaatgccaggtctcggacctggaatccgccacccgcattagaaccagggttgacatcggctcgaagcagtc

SEQ ID NO: 49 - DNA, 76 bp, Artificial
  Aptamer
gggtcaatgccaggtctcggacctggaatccgccacccgcattagaaccagggttgacatcggctcggaagcagtc

SEQ ID NO: 50 - DNA, 76 bp, Artificial
  Aptamer
gggtcaatgccaggtctcggacctggaatccgccacccgcattagaaccagggttgacatcggctcgcaaggagtc

SEQ ID NO: 51 - DNA, 76 bp, Artificial
  Aptamer
gggtcaatgccaggtctcggacctggaatccaccacccgcattagaaccagggttgacatcggctcgcaagcagtc

SEQ ID NO: 52 - DNA, 76 bp, Artificial
  Aptamer
gggtcaatgccaggtctcggacctggaatccgccacccgcattagaaccggggttgacatcggctcgcaagcagtc

SEQ ID NO: 53 - DNA, 75 bp, Artificial
  Aptamer
gggtcaatgccaggtctcggacctggaatccgccacccgcattagaaccagggttgacatcggctcgcaacagtc

SEQ ID NO: 54 - DNA, 76 bp, Artificial
  Aptamer
gggtcaatgccaggtctcgaacctggaatccgccacccgcattagaaccagggttgacatcggctcgcaagcagtc

SEQ ID NO: 55 - DNA, 76 bp, Artificial
  Aptamer
gggtcaatgccaggtctcgtacctggaatccgccacccgcattagaaccagggttgacatcggctcgcaagcagtc

SEQ ID NO: 56 - DNA, 76 bp, Artificial
  Aptamer
gggtcaatgccaggtctcggacctggaatccgccacccgcattagatccagggttgacatcggctcgcaagcagtc

SEQ ID NO: 57 - DNA, 76 bp, Artificial
  Aptamer
gggtcaatgccaggtctcggacctggaatccgccacccgcattaggaccagggttgacatcggctcgcaagcagtc

SEQ ID NO: 58 - DNA, 76 bp, Artificial
  Aptamer
gggtcaatgccaggtctcaactttcgcgtgtggttggtagggctaggtgtatacgcatatcggctcgcaagcagtc

SEQ ID NO: 59 - DNA, 75 bp, Artificial
  Aptamer
gggtcaatgccaggtctcaggtgagtcttacggatgtggttggtagggctaggtgtatatcggctcgcaagcagt

SEQ ID NO: 60 - DNA, 76 bp, Artificial
  Aptamer
gggtcaatgccaggtctctgcgtacttatctcgatgtggttggtagggataggtgtatatcggctcgcaagcagtc

SEQ ID NO: 61 - DNA, 74 bp, Artificial
  Aptamer
gggtcaatgccaggtctcgatctctgtggttggtaggggagtgtgtatagtgtcgtatcggctcgcaagcagtc

SEQ ID NO: 62 - DNA, 75 bp, Artificial
  Aptamer
gggtcaatgccaggtctcgctgtggttggtagggataggtgtatggcagctggataggatcggctcgcaagcagt

SEQ ID NO: 63 - DNA, 75 bp, Artificial
  Aptamer
gggtcaatgccaggtctcaataaaagtagctgatgtgcgttggtagggaacggtgtatatcggctcgcaagcagt

SEQ ID NO: 64 - DNA, 75 bp, Artificial
  Aptamer
gggtcaatgccaggtctcggcgtagaagccggaatgtggttggtagggttaggtgtatatcggctcgcaagcagt

SEQ ID NO: 65 - DNA, 76 bp, Artificial
  Aptamer
gggtcaatgccaggtctcgagccggtgttgtgttatgttggtagggaaggtgtataatatcggctcgcaagcagtc

SEQ ID NO: 66 - DNA, 58 bp, Artificial
  Core sequence A.5-1.9
gggtcaatgccaggtctcggacctggaatccgccacccgcattagaaccagggttgac

SEQ ID NO: 67 - DNA, 34 bp, Artificial
  Core sequence A5-2.9
cgcgtgtggttggtagggctaggtgtatacgcat

SEQ ID NO: 68 - DNA, 40 bp, Artificial
  Central region
aggtgagtcttacggatgtggttggtagggctaggtgtat

SEQ ID NO: 69 - DNA, 40 bp, Artificial
  Central region
tgcgtacttatctcgatgtggttggtagggataggtgtat

SEQ ID NO: 70 - DNA, 38 bp, Artificial
  Central region
gatctctgtggttggtaggggagtgtgtatagtgtcgt

SEQ ID NO: 71 - DNA, 40 bp, Artificial
  Central region
gctgtggttggtagggataggtgtatggcagctggatagg

SEQ ID NO: 72 - DNA, 40 bp, Artificial
  Central region
aataaaagtagctgatgtgcgttggtagggaacggtgtat

SEQ ID NO: 73 - DNA, 40 bp, Artificial
  Central region
ggcgtagaagccggaatgtggttggtagggttaggtgtat

SEQ ID NO: 74 - DNA, 40 bp, Artificial
  Central region
gagccggtgttgtgttatgttggtagggaaggtgtataat

SEQ ID NO: 75 - DNA, 18 bp, Artificial
  Primer
gggtcaatgccaggtctc

SEQ ID NO: 76 - DNA, 18 bp, Artificial
  Primer
atcggctcgcaagcagtc

SEQ ID NO: 77 - DNA, 10 bp, Artificial
  consensus moiety
gttggtaggg

SEQ ID NO: 78 - DNA, 7 bp, Artificial
  Consensus moiety
ggtgtat

SEQ ID NO: 79 - DNA, 3 bp, Artificial
  consensus moiety
tgt

SEQ ID NO: 80 - DNA, 56 bp, Artificial
  Aptamer
gtcaatgccaggtctcggacctggaatccgccacccgcattagaaccagggttgac

SEQ ID NO: 81 - DNA, 53 bp, Artificial
  Aptamer
gtcaatgccaggtctccctggaatccgccacccgcattagaaccagggttgac

SEQ ID NO: 82 - DNA, 50 bp, Artificial
  Aptamer
gtcaatgccagtcgctggaatccgccacccgcattagaaccagggttgac

SEQ ID NO: 83 - DNA, 52 bp, Artificial
  Aptamer
gtcaatgccaggtctcctggaatccgccacccgcattagaaccagggttgac

SEQ ID NO: 84 - DNA, 52 bp, Artificial
  Aptamer
gtcaatgccaggtcacctggaatccgccacccgcattagaaccagggttgac

SEQ ID NO: 85 - DNA, 53 bp, Artificial
  Aptamer
gtcaatgccaggtcgacctggaatccgccacccgcattagaaccagggttgac

SEQ ID NO: 86 - DNA, 52 bp, Artificial
  Aptamer
gtcaatgccaggtcgcctggaatccgccacccgcattagaaccagggttgac

SEQ ID NO: 87 - DNA, 54 bp, Artificial
  Aptamer
gtcaatgccaggtctcacctggaatccgccacccgcattagaaccagggttgac

SEQ ID NO: 88 - DNA, 54 bp, Artificial
  Aptamer
gtcaatgccaggtcggacctggaatccgccacccgcattagaaccagggttgac

SEQ ID NO: 89 - DNA, 55 bp, Artificial
  Aptamer
gtcaatgccaggtctcggacctggaatccgccacccgcattagaaccagggttga

SEQ ID NO: 90 - DNA, 55 bp, Artificial
  Aptamer
gtcaatgccaggtctcggacctggaatccgccacccgcatagaaccagggttgac

SEQ ID NO: 91 - DNA, 53 bp, Artificial
  Aptamer
gtcaatgccagtcggacctggaatccgccacccgcattagaaccagggttgac

SEQ ID NO: 92 - DNA, 55 bp, Artificial
  Aptamer
gtcaatgccaggtctcggacctggaatccgccacccgcttagaaccagggttgac

SEQ ID NO: 93 - DNA, 52 bp, Artificial
  Aptamer
gtcaatgccagcggacctggaatccgccacccgcattagaaccagggttgac

SEQ ID NO: 94 - DNA, 40 bp, Artificial
  central region
ggacctggaatccgccacccgcattagaaccagggttgac

SEQ ID NO: 95 - DNA, 40 bp, Artificial
  central region
aactttcgcgtgtggttggtagggctaggtgtatacgcat